(19) United States
(12) Patent Application Publication
(10) Pub. No.: US 2011/0136124 A1
(43) Pub. Date: Jun. 9, 2011

(54) MICRORNA EXPRESSION PROFILES ASSOCIATED WITH LUNG CANCER

(75) Inventors: Wilson ROA, Edmonton (CA); James XING, Edmonton (CA)

(73) Assignee: Wilson ROA, Edmonton, AB (CA)

(21) Appl. No.: 12/888,745

(22) Filed: Sep. 23, 2010

Related U.S. Application Data
(60) Provisional application No. 61/245,116, filed on Sep. 23, 2009.

(51) Int. Cl. C12Q 1/68

(52) U.S. Cl. 435/6.12

(57) ABSTRACT

The present invention is directed to sputum microRNA expression profiles associated with lung cancer and methods of using same for screening a subject for the disease.

### FIELD OF THE INVENTION

This invention relates to microRNA expression profiles associated with lung cancer and methods of using such profiles for diagnosing or detecting cancerous lung tissue.

### BACKGROUND OF THE INVENTION

Lung cancer, which is characterized by uncontrolled cell growth in tissues of the lung, is the leading cause of cancer-related death in men and the second most common in women after breast cancer. Cancer originating from lung cells is regarded as a primary lung cancer and can start in the bronchi or in the alveoli. Cancer may also metastasize to the lung from other parts of the body. The two main types of lung cancer are non-small cell lung carcinoma (NSCLC) and small cell lung carcinoma (SCLC). NSCLC grows slower than SCLC and comprises all the lung carcinomas except small cell carcinoma, and includes adenocarcinoma of the lung, large cell carcinoma, and squamous cell carcinoma. SCLC (also known as oat cell carcinoma) is aggressive and refers to a form of bronchogenic carcinoma seen in the wall of a major bronchus, usually in a middle-aged person with a history of tobacco smoking. By the time most patients are diagnosed with either type, the cancer has metastasized to other parts of the body.

Current diagnostic tests for patients exhibiting symptoms of lung cancer (i.e., persistent cough, shortness of breath, blood in sputum) include chest X-rays to detect shadows or large lung tumors; computed tomography (CT) or PET-CT scans which can detect small tumors which are not visible on chest X-rays; and magnetic resonance imaging, bone marrow scan or biopsy to determine whether the cancer has spread. To confirm diagnosis, a sample of tissue is often obtained directly from the tumor using invasive techniques such as, for example, bronchoscopy, needle biopsy, thoracotomy, and mediastinoscopy. In rare cases, sputum can be easily obtained from coughing and examined cytologically to detect lung cancer since it contains exfoliated airway epithelial cells from the bronchial tree, including cancer cells. Various studies have demonstrated that sputum can be used to identify cells bearing tumor-related aberrations (Thunnissen, 2003; Li et al., 2007; Qiu et al., 2008). However, sputum cytology is limited by low specificity and sensitivity, and subjectivity due to reliance on interpretation by cytopathologists.

Advances in molecular genetics have enabled the identification of genetic markers which are associated with cancer and may serve as useful tools for diagnostic or prognostic methods. MicroRNAs (miRNAs) are a class of single-stranded non-coding RNA molecules of about 19-25 nucleotides in length. MicroRNAs have been implicated in the control of many fundamental cellular and physiological processes including tissue development, cellular differentiation and proliferation, metabolic and signaling pathways, apoptosis, stem cell maintenance, cellular transformation and carcinogenesis. Particular miRNAs abnormally expressed in several types of cancer include for example, miR-155 which is upregulated in breast, colon and lung cancer; miR-92 which is downregulated in six solid cancer types by PAM (Volinia et al., 2006); hsa-let-7a which is downregulated in lung cancer and breast cancer (Yanaihara et al., 2006; Johnson et al., 2005; Iorio et al., 2005); and miR-9 which is increased in breast cancer and downregulated in lung cancer (Iorio et al., 2005; Yanaihara et al., 2006). miR-17-5p is expressed in breast, colon, lung, pancreas and prostate cancers. miR-21 is expressed in most solid cancer cells but not non-cancerous tissue. miR-143 and miR-145 are expressed in all cancerous tissues except stomach cancer tissue. hsa-miR-205 is a known marker for squamous cell lung carcinoma. miRNAs are commonly shared among different cancer histotypes. However, it is difficult to rely upon a single miRNA to identify a specific type of cancer since the miRNA may be expressed in several cancer types.

Lung cancer mortality is particularly high due to the lack of effective screening. Screening tests detect the possibility that a cancer is present before symptoms occur, but usually are not definitive, costly, and have psychological or physical repercussions in the event that false-positive or false-negative results are obtained. Screening using current techniques has not been shown to improve lung cancer survival.

### SUMMARY OF THE INVENTION

The present invention relates to sputum microRNA expression profiles associated with lung cancer and methods of using microRNA expression profiles for screening a subject for the disease, and monitoring progression of the disease in a subject.

In one aspect, the invention comprises a method of screening a subject for lung cancer comprising the steps of:
a) obtaining a sputum sample from the subject; and
b) determining a subject microRNA expression profile from the sputum sample;
c) determining whether or not the subject has lung cancer by determining a measure of similarity or dissimilarity of the subject expression profile to at least one known lung cancer microRNA expression profile and a known control microRNA expression profile;
   wherein each of the subject and known expression profiles comprise the expression levels of at least two microRNAs.

In one embodiment, the method may be used to monitor progression of the disease in a subject who has undergone treatment for the disease.

In one embodiment, the lung cancer is a non-small cell lung carcinoma which is resistant to radiation and drugs. In one embodiment, the lung cancer is a non-small cell lung carcinoma which is sensitive to radiation and drugs. In one embodiment, the lung cancer may be small cell lung carcinoma, or lung cancer which has metastasized from primary carcinomas of the breast, prostate, brain, or other tissue.

In one embodiment, the microRNA expression profile comprises the expression level of at least two of miR-21, miR-92, miR-143, miR-145, miR-155, miR-210, miR-17-5p, hsa-let-7a, hsa-miR-182, hsa-miR-205, or hsa-miR-372.

In one embodiment, the microRNA expression profile comprises the expression level of at least two of miR-21, miR-155, miR-210, miR-143, or hsa-miR-372.

In one embodiment, the microRNA expression profile comprises the expression level of either miR-145 or hsa-miR-205, or both.

In one embodiment, the step of determining the miRNA expression profile comprises a real-time quantitative polymerase chain reaction (RT-PCR) assay. In one embodiment, the comparison step comprises the step of comparing the miRNA expression profile obtained from the sputum sample with microRNA expression profiles obtained from normal epithelial cells, normal lung fibroblast, or cancer cells that are non-lung cancer cells. In one embodiment, the non-lung cancer cells are selected from breast cancer, prostate cancer, or glioblastoma cells.

In one embodiment, the determination of similarity or dissimilarity step comprises grouping the subject microRNA expression profile with other expression profiles from lung cancer cells, or control cells, or both, according to similarity of the expressed microRNAs and determining whether the expression profile of the subject falls into a group. In one embodiment, the grouping comprises the step of creating a cluster diagram. In one embodiment, the cluster diagram comprises a dendrogram.

In another aspect, the invention may comprise a method of monitoring progress of a subject undergoing treatment for lung cancer, comprising the steps of determining a subject microRNA expression profile from a sputum sample from the subject obtained post-treatment and determining a measure of similarity or dissimilarity of the subject expression profile to at least one known lung cancer microRNA expression profile, a known control microRNA expression profile, or the subject microRNA expression profile pre-treatment.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings:

FIG. 1 shows amplification curves for miRNAs obtained from a normal sputum sample.

FIG. 2 shows amplification curves for a mixture of normal sputum and A549 cells.

FIG. 3A shows the amount of RNA amount (μg) in sputum during storage at −20° C. over fourteen days.

FIG. 3B shows the relative expression of miRNAs (miR-21, miR-92 and U6) in sputum samples during storage at −20° C. over fourteen days.

FIG. 3C shows the relative expression of miRNAs (miR-21, miR-92 and U6) in sputum samples spiked with A549 cells (10⁵ cells in 200 μl sputum) during storage at −20° C. over fourteen days.

FIG. 4A shows an amplification curve of miR-21 in A549 cells extracted from sputum samples. FIG. 4B shows a standard curve of miR-21 for GM38 (normal epithelium fibroblast), A549 (non-small cell lung carcinoma), and MCF-7 (breast cancer) cells.

FIGS. 5A-F show miRNA profiles for different types of cancers: (A) plot showing miRNA expression for different cell lines and miRNAs; (B) A549 cells (non-small cell lung carcinoma); (C) mes-1 cells (non-small cell lung carcinoma); (D) MCF-7 cells (breast cancer); (E) Du145 cells (prostate cancer); and (F) U118 cells (glioblastoma).

FIG. 6 is a dendrogram showing hierarchical clustering based on the fold of miRNA expression of the cell lines.

FIG. 7 shows miRNA amplification curves for normal and cancer cell lines.

FIG. 8 shows miRNA expression of normal (GM38) and cancer (A549, H460, H1792, mes-1, U118) cell lines.

FIG. 9 is a dendrogram showing hierarchical clustering based on the fold of miRNA expression of normal (GM38) and cancer (A549, H460, H1792, mes-1, U118) cell lines.

FIG. 10 shows the amount of RNA (μg) in sputum samples from subjects designated as D1 and D2 (both cancer); D3 (successfully treated for cancer); D4 (cancer-free); and J16 (smoker control).

FIG. 11 shows the relative quantity of selected miRNAs in sputum samples from the subjects of FIG. 10.

FIG. 12 shows the miRNA expression profiles of sputum samples from the subjects of FIG. 10.

FIG. 13 is a dendrogram showing hierarchical clustering based on relatedness of selected miRNAs.

FIG. 14 is a dendrogram showing hierarchical clustering based on miRNA expression profiles (i.e., miR-21, miR-155, miR-210, miR-143, and hsa-miR-372) of the sputum samples of the subjects of FIG. 10.

FIG. 15 shows the miRNA expression profiles in sputum samples from subjects designated as D1, D2, D6, D7 (cancer); D4, D5 (normal); J16 (normal smoker); and SA-27 (normal smoker saliva).

FIG. 16 is a dendrogram showing hierarchical clustering based on the relatedness of the sputum samples of the subjects of FIG. 15.

### DETAILED DESCRIPTION OF PREFERRED EMBODIMENTS

When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

To facilitate understanding of the invention, the following definitions are provided.

The term “microRNA” abbreviated as “miRNA” means a class of non-coding RNA molecules of about 19-25 nucleotides derived from endogenous genes which act as post-transcriptional regulators of gene expression. They are processed from longer (ca 70-80 nt) hairpin-like precursors termed pre-miRNAs by the RNAse III enzyme Dicer. miRNAs assemble in ribonucleoprotein complexes termed “miRNPs” and recognize their target sites by antisense complementarity, thereby mediating down-regulation of their target genes. Near-perfect or perfect complementarity between the miRNA and its target site results in target mRNA cleavage, whereas limited complementarity between the miRNA and the target site results in translational inhibition of the target gene.

The term “non-small cell lung carcinoma” abbreviated as “NSCLC” means a group of lung cancers comprising all the carcinomas except small cell carcinoma, and including adenocarcinoma of the lung, large cell carcinoma, and squamous cell carcinoma. As used herein, the terms “cancer” and “carcinoma” are synonymous and may be used interchangeably.

The term “small cell lung carcinoma” abbreviated as “SCLC” means a common, highly malignant type of lung cancer, a form of bronchogenic carcinoma seen in the wall of a major bronchus, usually in a middle-aged person with a history of tobacco smoking.

The term “sputum” means material (for example, mucus or phlegm) which is expectorated or sampled from the respiratory tract.

The term “threshold cycle” or “C_{T}” means the fractional cycle number at which fluorescence has passed the fixed threshold.

In one embodiment, the present invention comprises sputum microRNA expression profiles which are associated with lung cancer. The expression profiles may be used to screen a subject for the disease. The microRNA expression profiles may be detected in sputum from a subject in order to discriminate lung cancer cells from epithelial or lung fibroblast cells; lung cancer from other cancer types; and between sub-types of lung cancer (i.e., either resistant or sensitive to radiation and drugs). The miRNA expression profiles disclosed herein are thus diagnostic and prognostic markers of lung cancer.

In one embodiment, the invention comprises a method of screening a subject for lung cancer comprising the steps of:
a) obtaining a sputum sample from the subject; and
b) determining a subject microRNA expression profile from the sputum sample;
c) determining whether or not the subject has lung cancer by determining a measure of similarity or dissimilarity of the subject expression profile to at least one known lung cancer microRNA expression profile and a known control microRNA expression profile;
   wherein each of the subject and known expression profiles comprise the expression levels of at least two microRNAs.

In one embodiment, the method may distinguish between types of lung cancer, by comparing the miRNA expression profiles to known expression profiles from, for example, a non-small cell lung carcinoma which is resistant to radiation and drugs, and/or a non-small cell lung carcinoma which is sensitive to radiation and drugs, and/or a small cell lung carcinoma, and/or a lung metastases originating from primary carcinomas of the breast, prostate, brain, or other tissue.

In one embodiment, the miRNA expression profile comprises the expression levels of at least two of miR-21, miR-92, miR-143, miR-145, miR-155, miR-210, miR-17-5p, hsa-let-7a, hsa-miR-182, hsa-miR-205, or hsa-miR-372. In one embodiment, the miRNA expression profile comprises the expression levels of miR-21, miR-155, miR-210, miR-143, and hsa-miR-372. In another embodiment, the miRNA expression profile comprises the expression levels of either miR-145 or hsa-miR-205, or both.

In one embodiment, the step of determining the miRNA expression profile comprises the use of a real-time quantitative PCR assay or micro-array analysis.

In one embodiment, the step of comparing the subject expression profile comprises grouping known microRNA expression profiles according to similarity of the expressed microRNAs and determining whether the subject expression profile is more similar to one group than the others. Similarity may be determined by statistical analysis, using methods known to one skilled in the art. In one embodiment, this grouping step comprises the step of creating a cluster diagram produced by hierarchical clustering. In one embodiment, the cluster diagram comprises a dendrogram.

miRNA profiling in sputum may be useful as a tool for cancer detection, classification, diagnosis and prognosis, since certain miRNA expression profiles can be correlated with certain cancers, or the absence of cancer. Thus, in the development of one embodiment of the present invention, it was determined whether a particular miRNA expression profile formed a signature or “barcode”, which may be indicative of cancer types and sub-types. Twelve miRNA candidates were selected including eleven miRNAs related to various cancer types and one endogenous control miRNA (U6):

TABLE 1
miRNA candidates for miRNA profiling

| | Mature miRNA | SEQ ID: | |
|---|---|---|---|
| miRNA | sequence | NO | Cancer type |
| miR-21 | UAGCUUAUCAG | 1 | solid cancer |
| | ACUGAUGUUGA | | cells |
| miR-92 | UAUUGCACUUG | 2 | solid cancer |
| | UCCCGGCCUG | | cells |
| miR-143 | UGAGAUGAAGC | 3 | all cancers |
| | ACUGUAGCUCA | | except |
| miR-145 | GUCCAGUUUUCC | 4 | stomach |
| | CAGGAAUCCCUU | | |
| miR-155 | UUAAUGCUAAU | 5 | lung, breast, |
| | CGUGAUAGGGG | | colon |
| miR-210 | CUGUGCGUGUG | 6 | lung, breast |
| | ACAGCGGCUGA | | |
| miR- | CAAAGUGCUUAC | 7 | lung, breast, |
| 17-5p | AGUGCAGGUAGU | | colon, pancreas, |
| | | | prostate |
| hsa-let- | UGAGGUAGUAG | 8 | lung (NSCLC), |
| 7a | GUUGUAUAGUU | | breast |
| hsa-miR- | UUUGGCAAUGG | 9 | lung (NSCLC) |
| 182 | UAGAACUCACA | | |
| hsa-miR- | UCCUUCAUUCC | 10 | squamous cell |
| 205 | ACCGGAGUCUG | | lung carcinoma |
| hsa-miR- | AAAGUGCUGCGA | 11 | lung (NSCLC) |
| 372 | CAUUUGAGCGU | | |
| U6 | GCAGGGGCCATGCTA | 12 | Control |
| | ATCTTCTCTGTATCG | | |

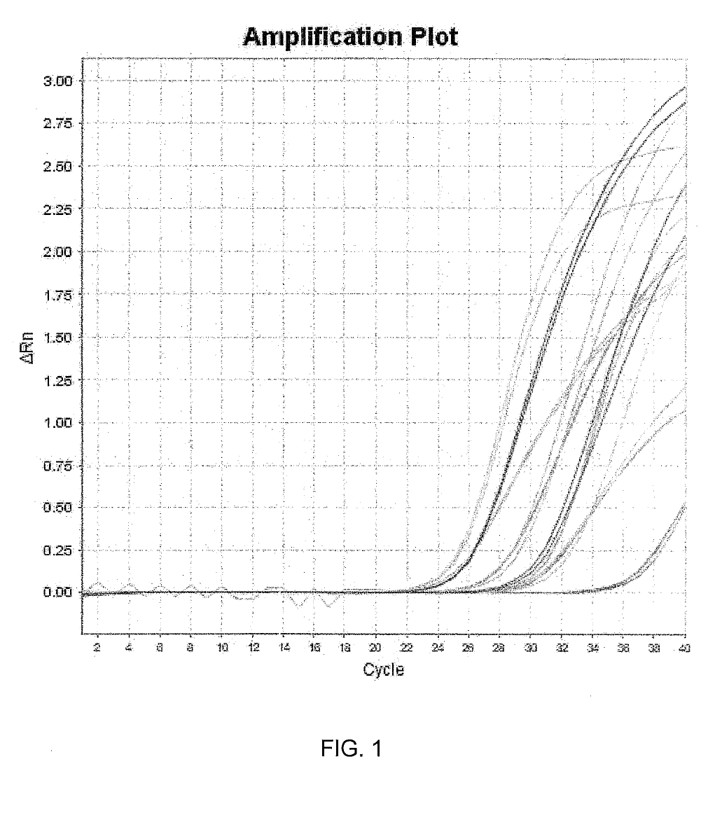

All twelve miRNAs in Table 1 exist in sputum from a subject without lung cancer. Table 2 shows the variation (ΔΔC_{T} SE) of duplicate samples which met the required amount for quantitative RT-PCR. FIG. 1 shows the amplification curve of the miRNAs in normal sputum.

TABLE 2
Accuracy and variation results of miRNAs in sputum

| | | | | | ΔC_{T}/ΔΔC_{T} |
|---|---|---|---|---|---|
| | C_{T} | C_{T} Mean | C_{T} SD | ΔC_{T}/miR-92 | SE |
| miR-21 | 25.138 | 25.113 | 0.034 | 0.917 | 0.103 |
| | 25.089 | | | | |
| miR-145 | 29.494 | 29.648 | 0.219 | 5.452 | 0.239 |
| | 29.803 | | | | |
| miR-155 | 30.156 | 30.163 | 0.009 | 5.967 | 0.097 |
| | 30.169 | | | | |
| hsa-miR-205 | 27.966 | 27.748 | 0.308 | 3.552 | 0.323 |
| | 27.530 | | | | |
| miR-210 | 30.420 | 30.129 | 0.411 | 5.933 | 0.422 |
| | 29.839 | | | | |
| U6 | 24.797 | 24.812 | 0.022 | 0.616 | 0.100 |
| | 24.828 | | | | |
| miR-92 | 24.264 | 24.196 | 0.097 | 0.000 | 0.137 |
| | 24.127 | | | | |
| miR-17-5p | 31.254 | 31.254 | | 7.058 | |
| miR-143 | 36.604 | 36.515 | 0.125 | 12.319 | 0.158 |
| | 36.427 | | | | |
| hsa-miR-182 | 30.638 | 30.638 | | 6.442 | |
| miR-392 | 36.532 | 36.744 | 0.299 | 12.548 | 0.315 |
| | 36.956 | | | | |
| hsa-let-7a | 27.425 | 27.425 | | 3.229 | |

The stability of miRNAs in sputum was determined since sputum may contain high levels of RNase activity. Endogenous miRNA, rather than naked exogenous miRNA, was used as a marker due to its resistance to RNase activity. A549 cells (lung cancer—NSCLC) were spiked into sputum samples collected from a healthy subject to mimic sputum of a subject with lung cancer. Mixtures of 50 μl of sputum with different numbers of A549 cells (0, 10², 10⁴, 10⁸) were used to determine the lowest detection limit (LOD), reproducibility and variation. As determined by UV, the LOD of A549 cells in sputum was 10² (Table 3). There is no linear relation between cell number and RNA concentration. From the difference (0.0723-0.0426) of 10⁴ and 10² cells, a very low LOD may be possible such as, for example, ten cells.

TABLE 3
UV results of miRNA extracted from A549-sputum mixture

| Cell + 50 μl | OD at | | | | |
|---|---|---|---|---|---|
| sputum | 260 nm | Mean OD | OD SD | Ratio 260/280 | RNA (μg) |
| 10⁶ | 0.2384 | 0.2364 | 0.0028 | 1.86 | 10.40 |
| | 0.2344 | | | | |
| 10⁴ | 0.1006 | 0.0982 | 0.0017 | 1.20 | 4.32 |
| | 0.0982 | | | | |
| 10² | 0.0734 | 0.0723 | 0.0015 | 1.21 | 3.18 |
| | 0.0712 | | | | |
| 0 | 0.0440 | 0.0426 | 0.0001 | 1.63 | 1.87 |
| | 0.0426 | | | | |

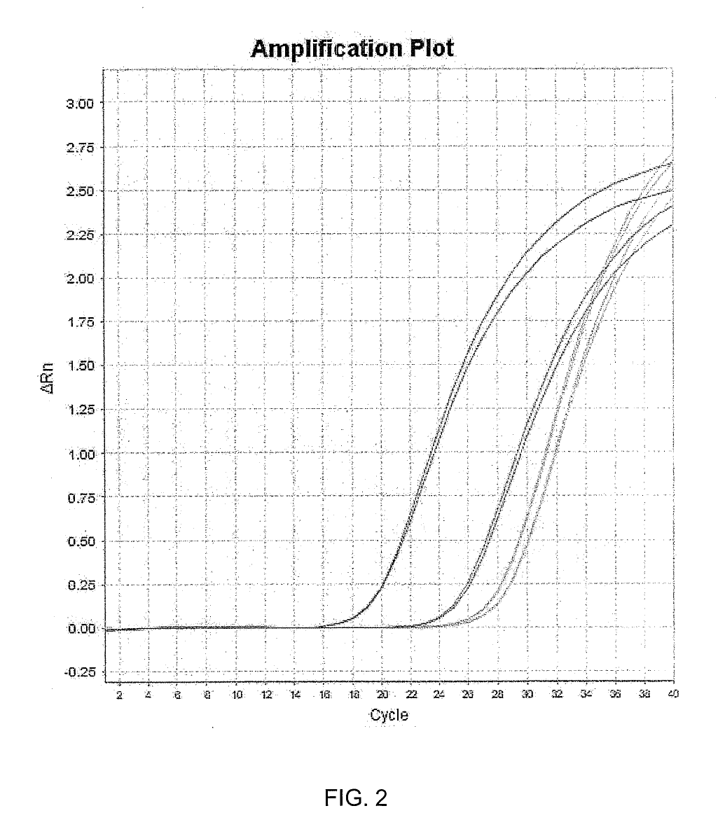

As determined by quantitative RT-PCR, the LOD of A549 cells in sputum was 10² for miR-21 (Table 4; FIG. 2). The LOD of A549 cells in sputum was 10⁴ for miR-92. The difference of LOD for different miRNA resulted from the different ambulance of miRNA (i.e., related cell type and miRNA) in the same cells. Accuracy and variation of quantitative RT-PCR experiments were optimal (minimum C_{T} SD 0.004). C_{T} was not linear with cell number in the mixtures. As with the UV detection, a similar result was obtained in regard to RNA amount and cell number. With an increase of cell number added in sputum, the C_{T} response decreases (equates to an increase in miRNA amount). Quantification of miR-21 expression by real-time RT-PCR showed that endogenous miRNA (miR-21) was clearly detected in the samples.

TABLE 4
miRNA detection limit from quantitative RT-PCR

| | Cell + 50 μl | | | | |
|---|---|---|---|---|---|
| | of sputum | C_{T} | C_{T} Mean | C_{T} SD | Average SD |
| miR-21 | 10⁶ | 20.14675 | 20.109 | 0.053384 | 0.053 |
| | | 20.07125 | | | |
| | 10⁴ | 26.13311 | 26.03885 | 0.133299 | |
| | | 25.9446 | | | |
| | 10² | 28.24446 | 28.26131 | 0.023825 | |
| | | 28.27815 | | | |
| | 0 | 28.87718 | 28.88025 | 0.004336 | |
| | | 28.88331 | | | |
| | LOD: Baseline-3 × SD = 28.883 (C_{T} for sputum) − 0.159 = | | | | |
| | 28.724 > 28.2611 | | | | |
| | (C_{T} for 10² cells) | | | | |
| | LOD for miR-21 is 10² in sputum. | | | | |
| miR-92 | 10⁶ | 19.83921 | 19.43922 | 0.565678* | 0.133611 |
| | | 19.03922 | | | |
| | 10⁴ | 23.8376 | 23.9915 | 0.21765 | |
| | | 24.1454 | | | |
| | 10² | 25.99367 | 25.99045 | 0.004549 | |
| | | 25.98723 | | | |
| | 0 | 26.18302 | 26.05671 | 0.178633 | |
| | | 25.93039 | | | |

LOD: Baseline-3 × SD = 26.057 (C_{T} for sputum) − 0.399 =
25.718 > 25.990 (C_{T} for 10² cells) > 23.991
LOD for miR-92 is 10⁴ in sputum.
*Noise spike: 1. bubbles in the reaction; 2. Evaporation during the denaturation step due to improper sealing or seal leaks

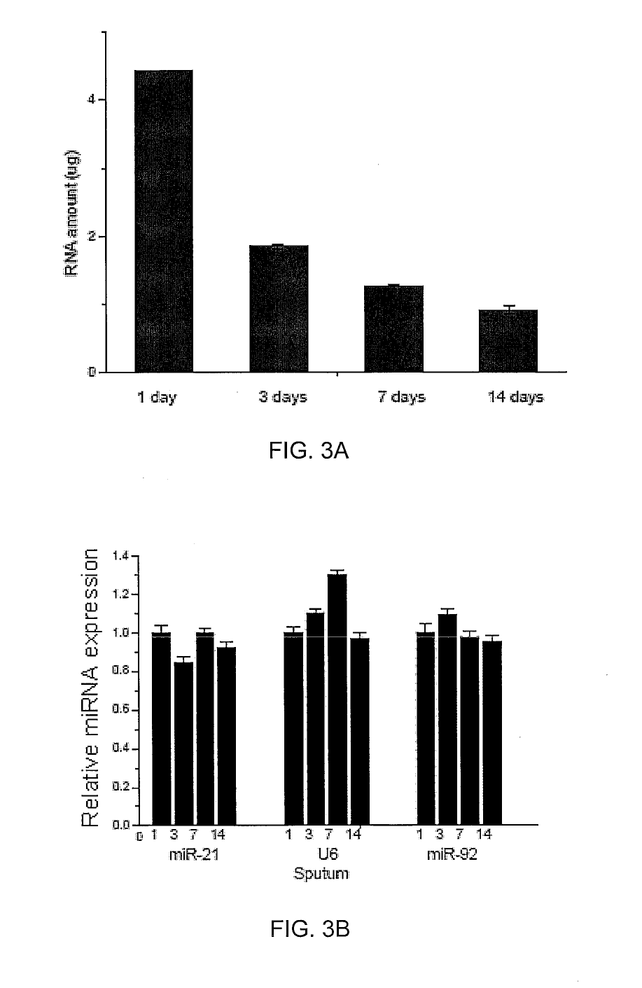

Sputum samples were stored at −20° C. for 1-14 days. As measured by UV-spectrometry, the RNA amount (μg) in sputum decreased over the fourteen day period, confirming that sputum contains high levels of RNase activity (FIG. 3A). However, there was no effect on expression levels of miR-21, miR-92 or U6 in sputum samples (FIG. 3B), or sputum samples spiked with A549 cells (10⁵ cells in 200 μl sputum) over the same period (FIG. 3C). The results indicate that endogenous miRNAs may be present in stable forms in sputum and reliably detected at various time points despite the presence of RNase activity.

miRNA expression profiles were determined in vitro using normal and cancer cell lines (American Type Culture Collection, Manassas, Va., USA) (Table 5), including four lung cancer cell lines. MES-1 and H1792 lung cancer cell lines are resistant to radiation and drugs in contrast to A549 and H460 lung cancer cell lines which are sensitive to such treatments.

TABLE 5
Cell lines and cancer type

| Cell Line | Cancer Type |
|---|---|
| A549 | Lung cancer (NSCLC) |
| MES-1 | Lung cancer (NSCLC) |
| H460 | Lung cancer (NSCLC) |
| H1792 | Lung cancer (adenocarcinoma) |
| MCF-7 | Breast cancer |
| DU145 | Prostate cancer |
| U118 | Glioblastoma |
| GM38 | Normal epithelium fibroblast |
| MRC-5 | Normal lung fibroblast |

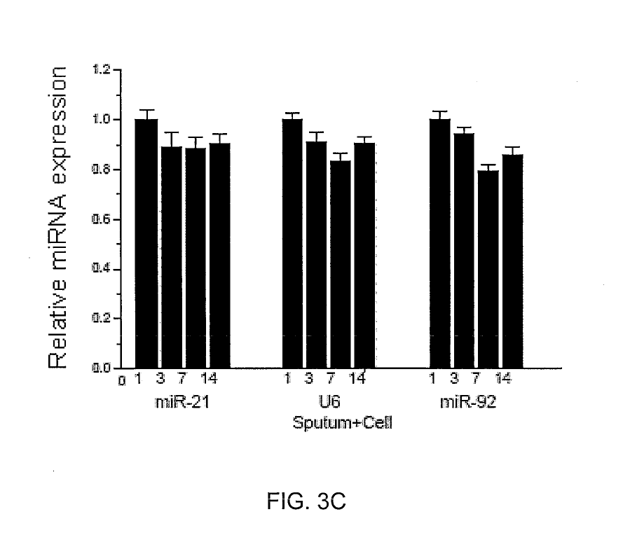

Initially, sputum samples collected from a healthy subject were spiked in vitro with cancer cells (A549 and MCF-7) to mimic the sputum of patients with cancer, and to validate the RT-PCR-based method described herein for determining miRNA expression profiles in sputum. GM38 cells (normal epithelium fibroblast) were used as the control. Briefly, 900 μl of sputum sample was added to duplicate 100 μl aliquots of each cell culture containing either 3, 16, 80, 400, 2000 or 10000 cells. The total RNA was extracted according to the procedure described in Example 1. The expression of miRNAs was determined by quantitative RT-PCR as described in Examples 4 and 5. The results confirmed linearity between the RNA input and the cycle threshold (Cₜ) values (FIGS. 4A and 4B). The miR-21 content among the three cell lines was greatest in MCF-7, followed by A549 and GM38 in that order. The assay had a dynamic range of at least six orders of magnitude (R²=0.9986), and was capable of detecting as few as three cells in the sputum samples (Table 6).

TABLE 6
Lowest detection limit for miRNAs in A549 cancer cells

| miRNA | miR-21 | miR-145 | miR-205 | miR-210 | U6 |
|---|---|---|---|---|---|
| LOD (cells) | <10 | 400 | 80 | <10 | <10 |

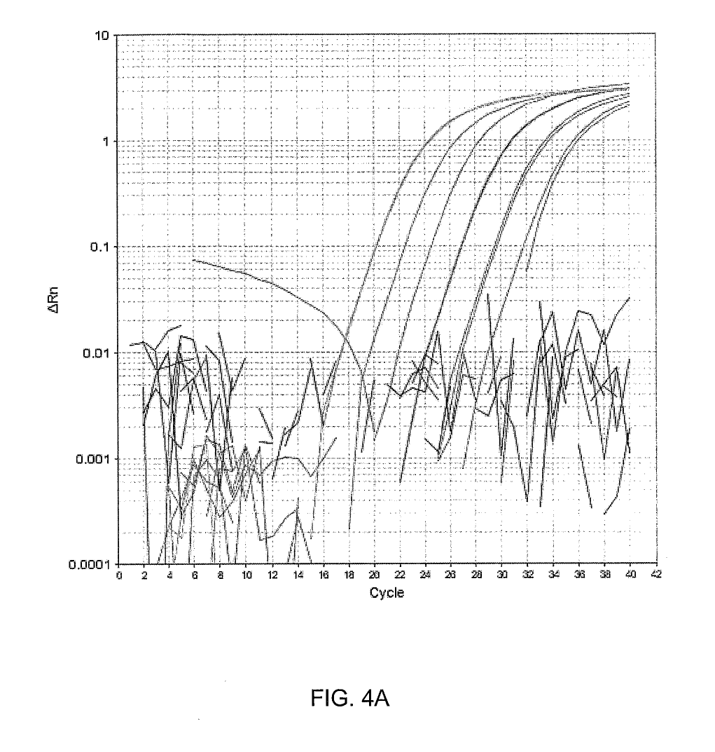

The combinations of miRNAs expressed in the cancer cell lines, or the controls, form a profile indicative of a specific cancer type, or the absence of cancer. miRNA expression profiles were obtained for A549 (lung carcinoma sensitive to radiation and drugs), mes-1 (lung carcinoma resistant to radiation and drugs), MCF-7 (breast cancer), Du145 (prostate cancer), and U118 (glioblastoma) cell lines (FIGS. 5A-5F). miR-145 was upregulated in A549 cells and downregulated in mes-1 cells. miR-145 thus shows potential as a marker in sputum to distinguish non-small cell lung carcinomas which are either sensitive (A549) or resistant (mes-1) to radiation and drugs. Further, hsa-miR-205, a highly specific marker for squamous cell lung carcinoma (NSCLC), was upregulated in A549 cells (FIG. 5B) and absent in Du145 cells (prostate cancer) (FIG. 5E).

In one embodiment, assessment of miRNA expression profiles was performed using hierarchical clustering which identifies relatively homogenous groups of cases or variables based on selected characteristics. In one embodiment, agglomerative hierarchical clustering was used which starts with each case as a cluster and combines new clusters until all individuals are grouped into one large cluster. Methods for combining clusters include, for example, between-group linkage, within-groups linkage, nearest neighbour, furthest neighbour, centroid clustering, median clustering, and Ward's method. In one embodiment, the method for combining clusters is between-group linkage. A convergence measure is used for measuring the similarity and divergence between cases (i.e., distance measuring). In one embodiment, the convergence measure is the Pearson correlation coefficient (denoted by r) which measures the correlation or linear dependence between two variables, giving a value between +1 and −1 inclusive. In one embodiment, a correlation of less than 0.90 may be considered as indicative of a significant difference.

The distance at which the clusters are combined may be presented graphically as a dendrogram which connects the cases based upon their similarity scores. The vertical lines show joined clusters. The position of the line on the scale indicates the distance at which clusters are joined. The observed distances are rescaled to fall into the range of 1 to 25; however, the ratio of the rescaled distances within the dendrogram is the same as the ratio of the original distances. Cases grouped on a lower distance are considered more similar than cases grouped at a higher distance.

Cluster analysis was performed, designating the cell line as “case” and the fold of miRNA expression profiles as “variable” (Example 6). Table 7 sets out a proximity matrix which presents the information for the distances between the cases (cell lines) and the clusters.

TABLE 7
Proximity matrix for cell lines

| | Correlation between vectors of values | | | | | | |
|---|---|---|---|---|---|---|---|
| Case (cell line) | A549 | mes-1 | Du145 | MCF-7 | U118 | GM38 | MRC-5 |
| A549 | 1.000 | 0.993 | 0.703 | 0.234 | −0.332 | −0.470 | 0.000 |
| mes-1 | 0.993 | 1.000 | 0.752 | 0.329 | −0.259 | −0.551 | 0.000 |
| Du145 | 0.703 | 0.752 | 1.000 | 0.825 | −0.359 | −0.940 | 0.000 |
| MCF-7 | 0.234 | 0.329 | 0.825 | 1.000 | 0.022 | −0.967 | 0.000 |
| U118 | −0.332 | −0.259 | −0.359 | 0.022 | 1.000 | 0.113 | 0.000 |
| GM38 | −0.470 | −0.551 | −0.940 | −0.967 | 0.113 | 1.000 | 0.000 |
| MRC-5 | 0.000 | 0.000 | 0.000 | 0.000 | 0.000 | 0.000 | 1.000 |

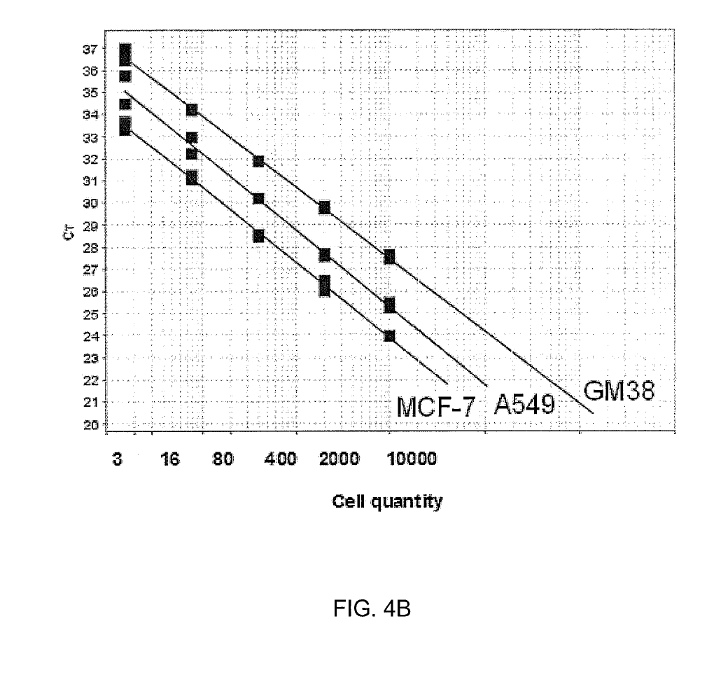

FIG. 6 is a dendrogram showing hierarchical clustering based on the fold of miRNA expression profiles of the cell lines. The seven cell lines are roughly separated into four main clusters. The first cluster contains the non-small cell lung carcinomas A549 (lung cancer sensitive to radiation and drugs) and mes-1 (lung cancer resistant to radiation and drugs). The second cluster contains Du145 (prostate cancer) and MCF-7 (breast cancer). MRC-5 (normal lung fibroblast) is its own cluster, clearly separated from all other clusters. A fourth cluster contains U118 (glioblastoma) and GM38 (normal epithelium fibroblast); however, as U118 and GM38 are grouped together at a higher distance, they are considered as less similar to each other. These results indicate that miRNA expression profiles can be used to discriminate between different types of cancer (for example, lung versus prostate and breast cancer).

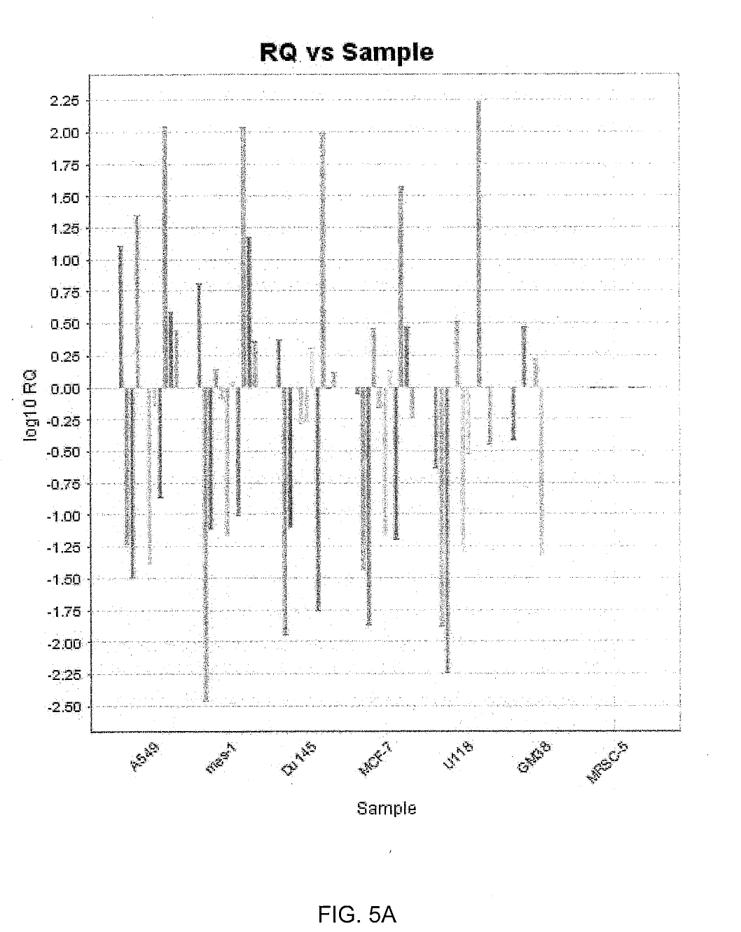

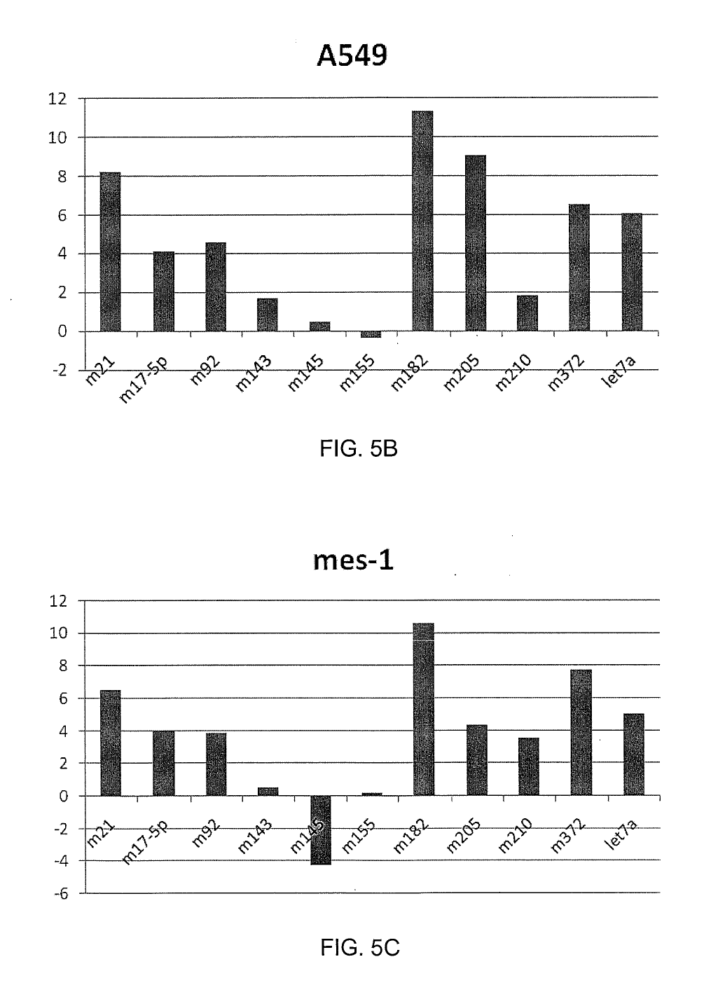

It was determined whether miRNA expression profiles may distinguish different sub-types of a cancer. The miRNA expression profiles of four different lung cell lines, namely A549 and H460 (both NSCLC sensitive to radiation and drugs), mes-1 (NSCLC resistant to radiation and drugs), and H1792 (adenocarcinoma resistant to radiation and drugs), were compared (FIGS. 7-9). The GM38 (normal epithelium fibroblast) cell line was included as a control, while the U118 (glioblastoma) cell line was included as representing a different cancer type (i.e., brain cancer). Cluster analysis was performed, designating the cell line as “case” and the fold of miRNA expression profile as “variable.” Table 8 sets out a proximity matrix which presents the information for the distances between the cases (cell lines) and the clusters.

TABLE 8
Proximity matrix for lung cell lines

| Case | Correlation between vectors of values | | | | | |
|---|---|---|---|---|---|---|
| (cell line) | A549 | H460 | H1792 | mes-1 | U118 | GM38 |
| A549 | 1.000 | 0.925 | 0.429 | 0.440 | 0.577 | 0.000 |
| H460 | 0.925 | 1.000 | 0.485 | 0.472 | 0.721 | 0.000 |
| H1792 | 0.429 | 0.485 | 1.000 | 0.709 | 0.607 | 0.000 |
| mes-1 | 0.440 | 0.472 | 0.709 | 1.000 | 0.141 | 0.000 |
| U118 | 0.577 | 0.721 | 0.607 | 0.141 | 1.000 | 0.000 |
| GM38 | 0.000 | 0.000 | 0.000 | 0.000 | 0.000 | 1.000 |

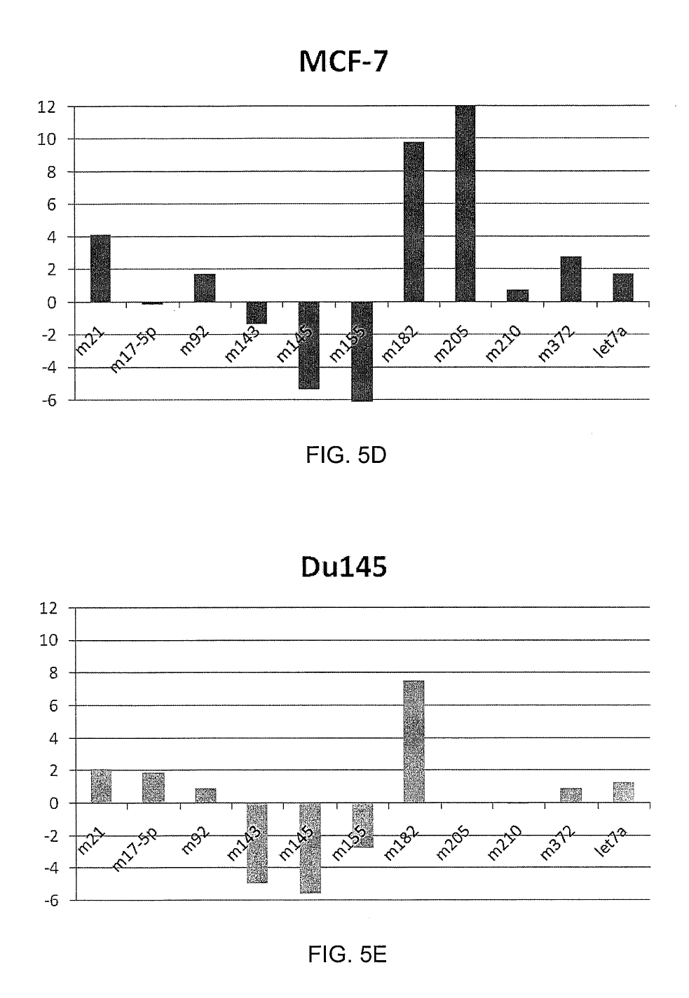

FIG. 9 is a dendrogram showing hierarchical clustering based on the fold of miRNA expression profiles of the cell lines. The six cell lines are roughly separated into four main clusters. The first cluster contains the lung carcinomas A549 and H460 (both NSCLC sensitive to radiation and drugs). U118 (glioblastoma) is its own cluster, clearly separated from all other clusters (similarity<0.9). The third cluster contains H1792 (adenocarcinoma resistant to radiation and drugs) and mes-1 (NSCLC resistant to radiation and drugs). GM38 (normal epithelium fibroblast) is its own cluster, clearly separated from all other clusters (similarity<0.9). These results indicate that miRNA expression profiles can be used to discriminate between different sub-types of cancer (for example, NSCLC which are either resistant or sensitive to radiation and drugs).

miRNA expression profiles obtained from sputum samples may be diagnostic and prognostic markers of lung cancer, by comparison with known expression profiles. miRNA expression profiles were determined using sputum samples collected from five subjects designated as D1 and D2 (both cancer); D3 (successfully treated for cancer); D4 (cancer-free); and J16 (smoker control) (Table 9).

TABLE 9
Data for sputum samples from subjects

| | Weight of | Weight of | Weight of | Mean | STD of | Density of |
|---|---|---|---|---|---|---|
| | 1^{st} 200 μl | 2^{nd} 200 μl | 3^{rd} 200 μl | weight | weights | Sputum |
| D1 | 0.2158 | 0.1948 | 0.2130 | 0.2079 | 0.0114 | 1.0393 |
| D2 | 0.2050 | 0.2054 | 0.1914 | 0.2006 | 0.0080 | 1.0030 |
| D3 | 0.2199 | 0.1983 | 0.2030 | 0.2071 | 0.0114 | 1.0353 |
| D4 | 0.2377 | 0.1673 | 0.2028 | 0.2026 | 0.0352 | 1.0130 |
| J16 | 0.2600 | 0.2516 | 0.2075 | 0.2397 | 0.0282 | 1.1985 |

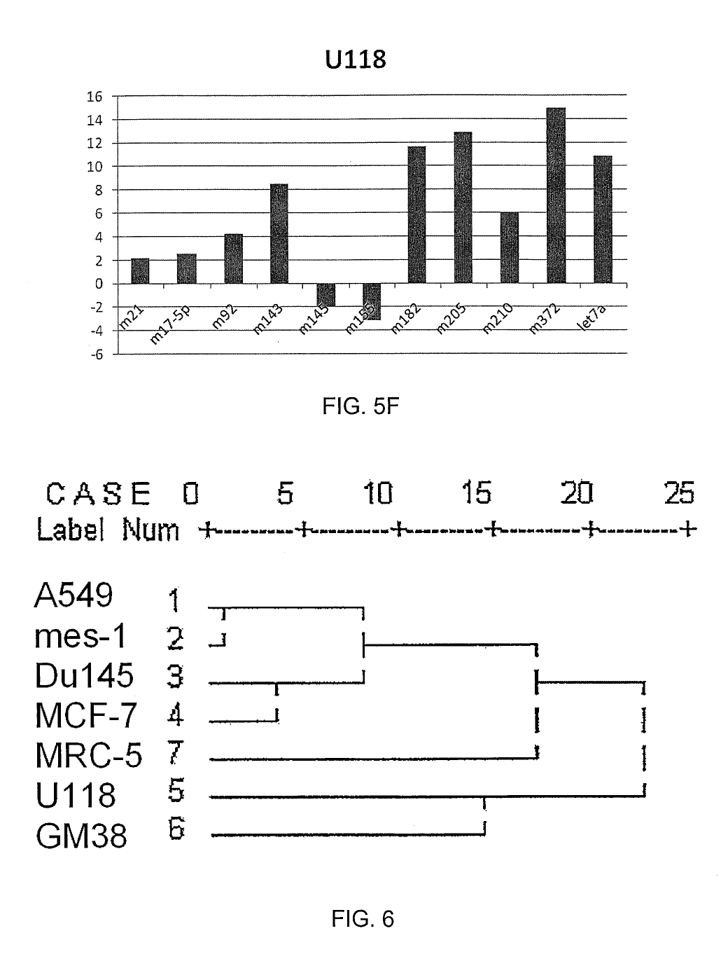

The sputum samples were then homogenized (Example 2) for RNA extraction as described in Example 3. The amount of RNA (μg) and RNA concentration in 200 μl of sputum sample from each subject was calculated (Example 3; FIG. 10; Table 10). FIG. 10 and Table 10 reflect the quantity of all RNA including miRNA which comprises only 0.01% of all RNA when extracted with the method described in Example 3.

TABLE 10
RNA concentration and quantity as determined using UV spectrophotometer

| | | | | | Concentration | | |
|---|---|---|---|---|---|---|---|
| | OD1 | OD2 | mean | STD | (ng/μl) | RNA (μg) | RNA STD |
| D1 | 0.2161 | 0.2252 | 0.2207 | 0.006 | 353.04 | 32.4796 | 0.4632 |
| D2 | 0.1393 | 0.141 | 0.1402 | 0.001 | 224.24 | 20.6300 | 0.0865 |
| D3 | 0.0501 | 0.0505 | 0.0500 | 0.0002 | 80.48 | 7.40416 | 0.0204 |
| D4 | 0.0499 | 0.0503 | 0.0500 | 0.0002 | 80.16 | 7.37472 | 0.0204 |
| J16 | 0.0453 | 0.0454 | 0.0907 | 7.07E−05 | 72.56 | 6.6755 | 0.0051 |

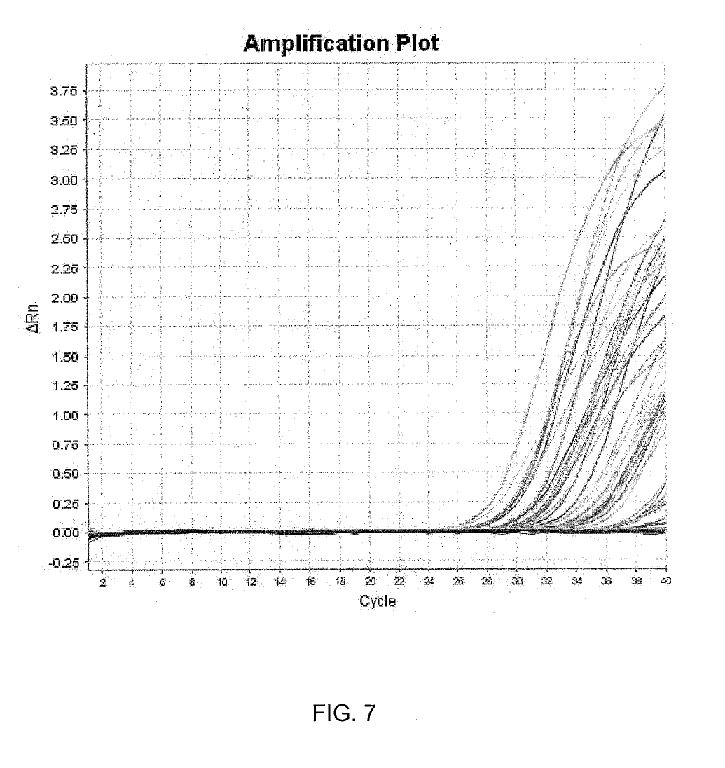

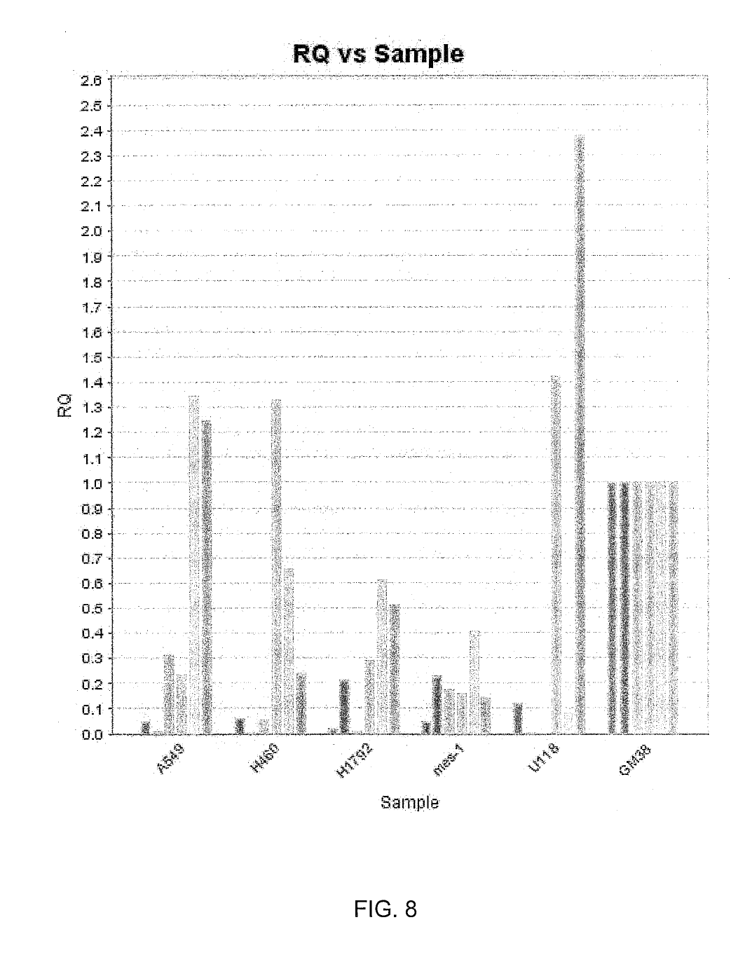

The relative quantity of selected miRNAs in 200 of sputum sample from each subject was determined using quantitative RT-PCR (FIG. 11; Examples 4 and 5). FIG. 12 and Table 11 show that the miRNA profiles of sputum samples from each subject appear to differ.

TABLE 11
miRNA expression in sputum samples

| | D1 | D2 | D3 | D4 | J16 |
|---|---|---|---|---|---|
| miR-21 | 1.7933 | 3.4762 | 0.6975 | 0.0000 | 1.0000 |
| miR-145 | 0.2438 | 6.1787 | 0.5133 | 0.0678 | 1.0000 |
| miR-155 | 1.0984 | 0.0090 | 0.3347 | 0.1046 | 1.0000 |
| hsa-miR-205 | 0.0481 | 0.1547 | 0.0141 | 0.4936 | 1.0000 |
| miR-210 | 0.0596 | 0.1528 | 0.1905 | 0.0702 | 1.0000 |
| miR-17-5p | 1.3634 | 3.2170 | 0.2932 | 0.3550 | 1.0000 |
| miR-143 | 0.7846 | 0.7470 | 0.0563 | 0.0484 | 1.0000 |
| hsa-miR-182 | 0.1985 | 0.4678 | 0.0251 | 0.0745 | 1.0000 |
| hsa-miR-372 | 0.0206 | 0.2305 | 0.4129 | 0.7435 | 1.0000 |
| hsa-let-7a | 0.0048 | 0.0101 | 0.0381 | 0.1170 | 1.0000 |

Table 12 sets out a proximity matrix which presents the information for the distances between the cases (miRNAs) and the clusters.

TABLE 12
Proximity matrix for miRNAs

| | Matrix File Input | | | | | | | | | |
|---|---|---|---|---|---|---|---|---|---|---|
| | miR- | miR- | miR- | hsa-miR- | miR- | miR-17- | miR- | hsa-miR- | hsa-miR- | hsa-let- |
| | 21 | 145 | 155 | 205 | 210 | 5p | 143 | 182 | 372 | 7a |
| miR-21 | 1.000 | −0.116 | −0.133 | 0.579 | −0.587 | 0.882 | −0.340 | 0.972 | 0.253 | −0.244 |
| miR-145 | −0.116 | 1.000 | 0.479 | 0.609 | 0.018 | −0.490 | 0.285 | −0.211 | 0.417 | 0.497 |
| miR-155 | −0.133 | 0.479 | 1.000 | 0.563 | 0.732 | −0.072 | 0.850 | −0.096 | 0.899 | 0.981 |
| hsa-miR-205 | 0.579 | 0.609 | 0.563 | 1.000 | −0.031 | 0.353 | 0.376 | 0.578 | 0.814 | 0.526 |
| miR-210 | −0.587 | 0.018 | 0.732 | −0.031 | 1.000 | −0.286 | 0.901 | −0.452 | 0.520 | 0.801 |
| miR-17-5p | 0.882 | −0.490 | −0.072 | 0.353 | −0.286 | 1.000 | −0.171 | 0.929 | 0.273 | −0.176 |
| miR-143 | −0.340 | 0.285 | 0.850 | 0.376 | 0.901 | −0.171 | 1.000 | −0.197 | 0.777 | 0.926 |
| hsa-miR-182 | 0.972 | −0.211 | −0.096 | 0.578 | −0.452 | 0.929 | −0.197 | 1.000 | 0.320 | −0.179 |
| hsa-miR-372 | 0.253 | 0.417 | 0.899 | 0.814 | 0.520 | 0.273 | 0.777 | 0.320 | 1.000 | 0.874 |
| hsa-let-7a | −0.244 | 0.497 | 0.981 | 0.526 | 0.801 | −0.176 | 0.926 | −0.179 | 0.874 | 1.000 |

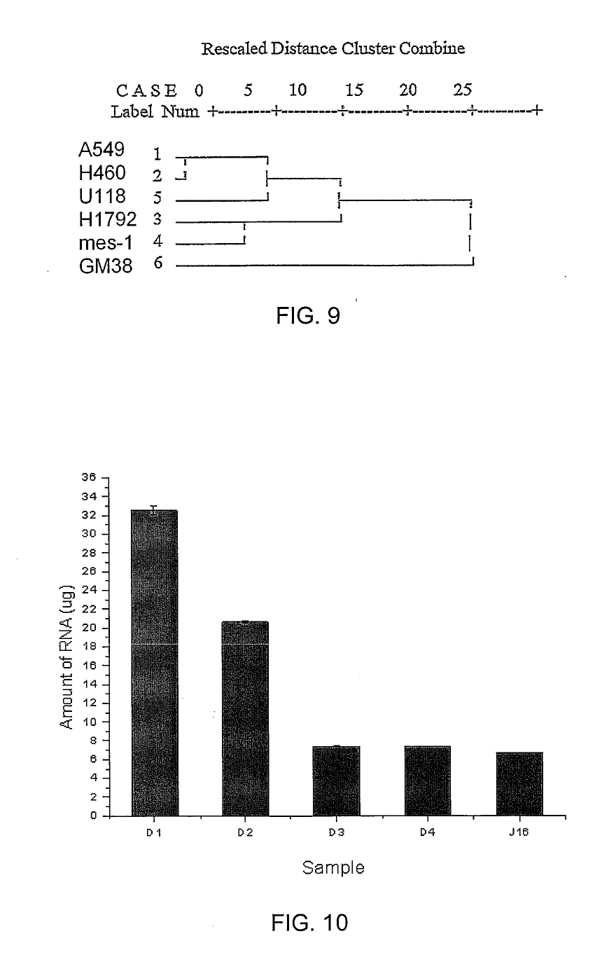

FIG. 13 is a dendrogram showing hierarchical clustering based on the relatedness of the miRNAs. The miRNAs are roughly separated into five main clusters. The first cluster contains miR-210 and hsa-let-7a. hsa-miR-182 is clearly separated from all other clusters. A third cluster contains hsa-miR-372 and hsa-miR-205. A fourth cluster contains miR-155 and miR-143. A fifth cluster contains miR-21, miR-17-5p and miR-145.

In one embodiment, miRNA expression profiles may comprise the expression level of at least two miRNAs, which are grouped in different clusters from each other.

From among the miRNAs analyzed above, five miRNAs (i.e., miR-21, miR-155, miR-210, miR-143, and hsa-miR-372) were selected for clustering analysis based on miRNA expression profiles of the sputum samples of the subjects. These miRNAs are from different clusters, except for miR-155 and miR-143, which are in the same cluster. Although hsa-miR-182 is in a separate cluster, it was not chosen as it is not associated with a lung cancer. Table 13 sets out a proximity matrix which presents the information for the distances between the cases (sputum samples of the subjects) and the clusters.

TABLE 13
Proximity matrix for sputum samples

| | Correlation between vectors of values | | | | |
|---|---|---|---|---|---|
| | D1 | D2 | D3 | D4 | J16 |
| D1 | 1.000 | 0.779 | 0.568 | −0.600 | 0.000 |
| D2 | 0.779 | 1.000 | 0.731 | −0.373 | 0.000 |
| D3 | 0.568 | 0.731 | 1.000 | 0.103 | 0.000 |
| D4 | −0.600 | −0.373 | 0.103 | 1.000 | 0.000 |
| J16 | 0.000 | 0.000 | 0.000 | 0.000 | 1.000 |

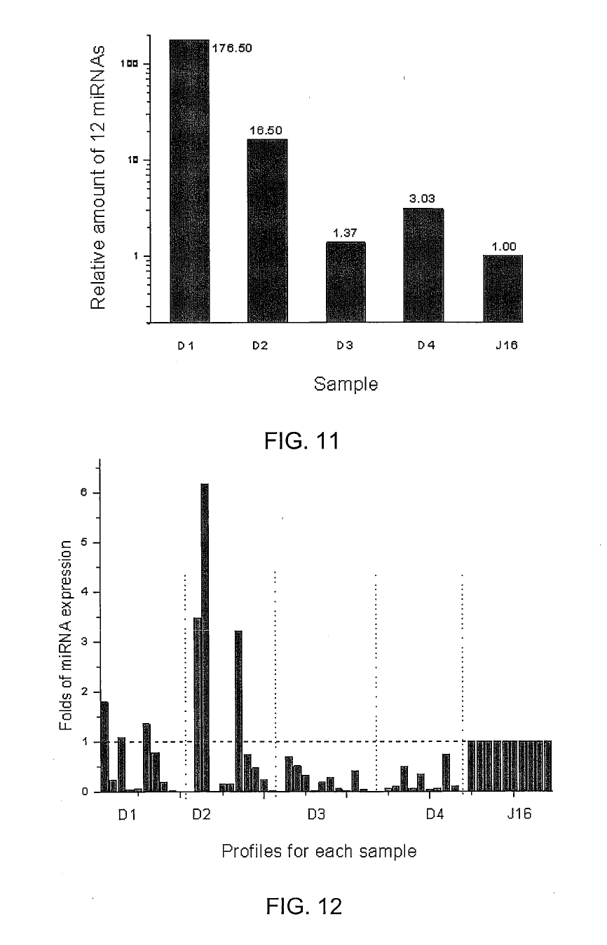

FIG. 14 is a dendrogram showing hierarchical clustering based on the relatedness of the sputum samples. The sputum samples are roughly separated into three main clusters. The first cluster contains D1 and D2 (both cancer) with the distance less than 0, indicating their high level of relatedness. D3 (treated for cancer) is separated from all other clusters. A third cluster contains D4 (cancer-free) and J16 (normal control), although the similarity between D4 and J16 is zero (Table 13). Overall, D1 and D2 show significant difference with D3, D4 and J16, indicating the miRNA expression profiles in sputum samples have potential in distinguishing between subjects with lung cancer and those without the disease.

In a further study, miRNA expression profiles were determined using sputum samples collected from subjects designated as D1, D2, D6, D7 (cancer); D4, D5 (normal); J16 (normal smoker); and SA-27 (normal smoker saliva). MRC-5 (normal lung fibroblast cell line) was used as the reference sample, and U6 as the endogenous control.

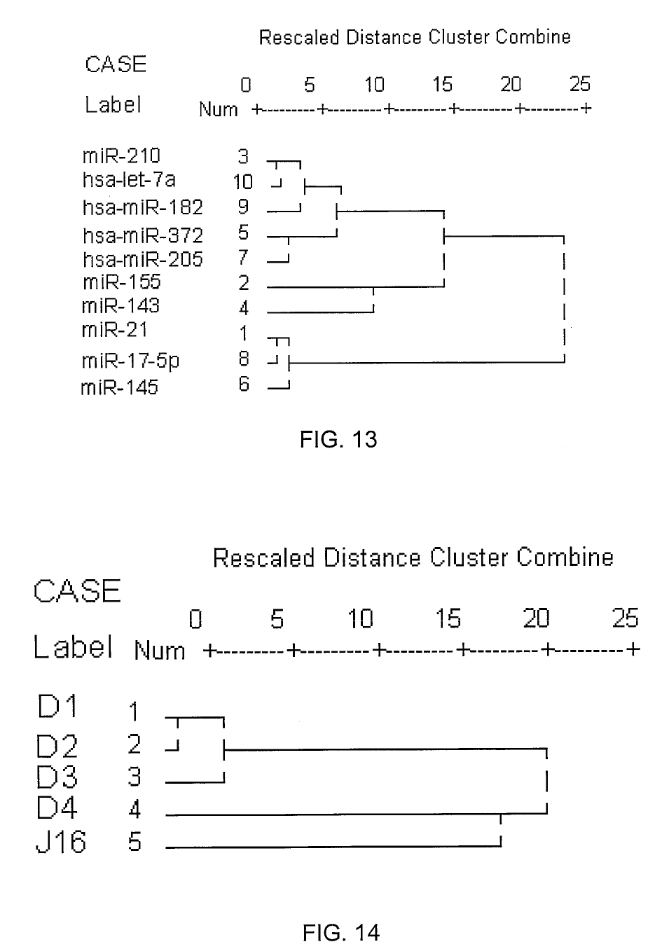

FIG. 15 and Table 14 show the miRNA expression profiles in the sputum samples from each subject. From among the miRNAs analyzed in Table 14, five miRNAs (i.e., miR-21, miR-155, miR-210, miR-143, and hsa-miR-372) were selected for clustering analysis based on miRNA expression profiles of the sputum samples of the subjects. Table 15 sets out a proximity matrix which presents the information for the distances between the cases (sputum samples of the subjects) and the clusters.

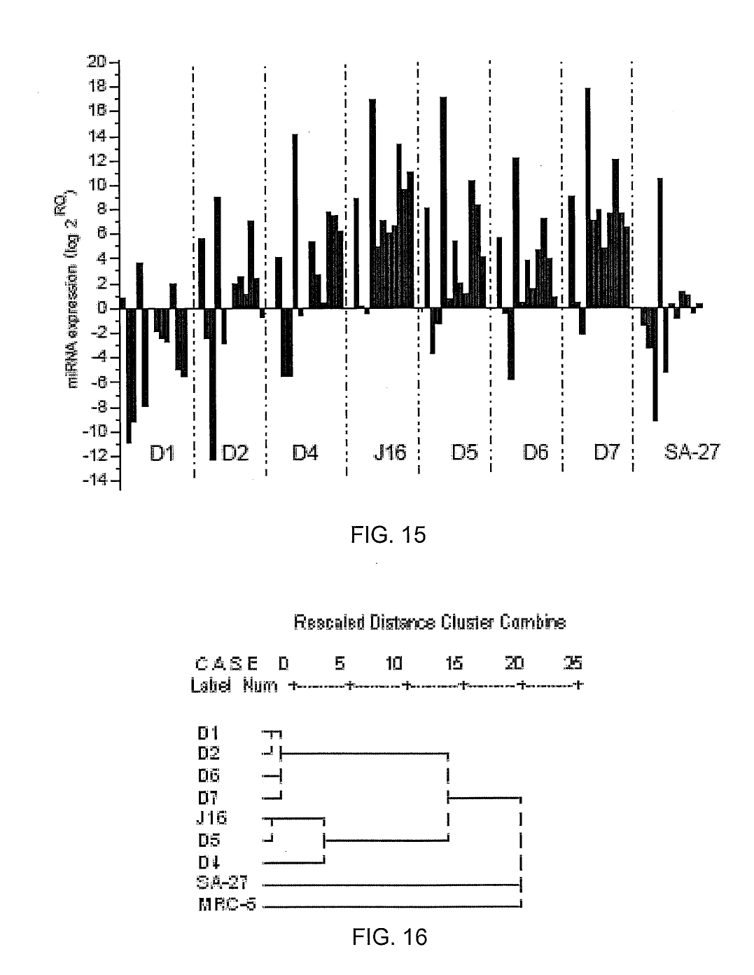

FIG. 16 is a dendrogram showing hierarchical clustering based on the relatedness of the expression profiles from the sputum samples. The expression profiles are roughly separated into four main clusters. The first cluster contains D1, D2, D6 and D7 (all cancer subjects), with the distance less than 0, indicating their high level of relatedness. The similarity of each pair is greater than 0.90 (i.e., minimum 0.901 (D1-D6) to maximum 0.993 (D1-D2)). A second cluster contains D4, D5 and J16 (non-cancer subjects). MRC-5 (normal lung fibroblast cell line) is separated from all other clusters. SA-27 (normal smoker saliva) is also separated from all other clusters. Table 16 summarizes the diagnostic results. Overall, the results indicate that the miRNA expression profiles in sputum samples have potential in distinguishing between normal and cancer subjects.

TABLE 14
miRNA expression fold (RQ)

| | | miR- | miR- | | | | miR- | | hsa-miR- | | |
|---|---|---|---|---|---|---|---|---|---|---|---|
| | miR-21 | 145 | 155 | hsa-miR-205 | miR-210 | miR-92 | 17-5p | miR-143 | 182 | miR-372 | hsa-let-7a |
| D1 | 1.7800 | 0.0005 | 0.0017 | 11.9800 | 0.0039 | 0.2444 | 0.1800 | 0.1600 | 3.9300 | 0.0300 | 0.0200 |
| D2 | 47.2700 | 0.1900 | 0.0002 | 529.1900 | 0.1400 | 3.9186 | 5.8300 | 2.0500 | 127.3500 | 5.1200 | 0.6000 |
| D4 | 0.0000 | 0.0200 | 0.0200 | 17227.5300 | 0.6400 | 39.9685 | 6.5700 | 1.3600 | 206.8900 | 168.5500 | 71.3000 |
| J16 | 472.0400 | 1.0500 | 0.7400 | 118788.8000 | 31.2600 | 136.1145 | 62.9500 | 95.4600 | 9451.6700 | 771.6600 | 2074.9900 |
| D5 | 267.6665 | 0.0745 | 0.3882 | 132601.0850 | 1.5981 | 40.5022 | 3.7671 | 2.1747 | 1177.3178 | 316.3120 | 16.0876 |
| D6 | 50.9739 | 0.7232 | 0.0180 | 4385.5530 | 1.2853 | 13.9040 | 2.7311 | 25.2940 | 149.8371 | 15.5262 | 1.7489 |
| D7 | 515.3364 | 1.3786 | 0.2315 | 223092.4810 | 130.7950 | 236.1950 | 28.3945 | 186.1718 | 4175.8021 | 198.2603 | 89.8809 |
| SA-27 | 0.3827 | 0.1088 | 0.0017 | 1303.4742 | 0.0270 | 1.1469 | 0.5421 | 2.4485 | 1.9381 | 0.7662 | 1.1826 |
| MRC-5 | 1.0000 | 1.0000 | 1.0000 | 1.0000 | 1.0000 | 1.0000 | 1.0000 | 1.0000 | 1.0000 | 1.0000 | 1.0000 |

TABLE 15
Proximity matrix for sputum samples

| | | Correlation between vectors of values | | | | | | | |
|---|---|---|---|---|---|---|---|---|---|
| | D1 | D2 | D4 | J16 | MRC-5 | D5 | D6 | D7 | SA-27 |
| D1 | 1.000 | 0.993 | −0.265 | 0.324 | 0.000 | 0.511 | 0.901 | 0.926 | −0.106 |
| D2 | | 1.000 | −0.162 | 0.418 | 0.000 | 0.601 | 0.893 | 0.936 | −0.152 |
| D4 | | | 1.000 | 0.825 | 0.000 | 0.691 | −0.083 | −0.025 | 0.029 |
| J16 | | | | 1.000 | 0.000 | 0.972 | 0.461 | 0.530 | 0.009 |
| MRC-5 | | | | 0.000 | 1.000 | 0.000 | 0.000 | 0.000 | 0.000 |
| D5 | | | | | | 1.000 | 0.572 | 0.658 | −0.118 |
| D6 | | | | | | | 1.000 | 0.947 | 0.305 |
| D7 | | | | | | | | 1.000 | 0.079 |
| SA-27 | | | | | | | | | 1.000 |

TABLE 16
Diagnostic results from cluster analysis of miRNA profiles

| | Subject | | | | | | | | |
|---|---|---|---|---|---|---|---|---|---|
| | D1 | D2 | D4 | J16 | MRC-5 | D5 | D6 | D7 | SA-27 |
| Sample | cancer | cancer | normal | Normal | Normal | normal | cancer | cancer | Normal |
| | | | | smoker | lung | | | | smoker |
| | | | | | cells | | | | saliva |

| | | | | | | |
|---|---|---|---|---|---|---|
| Diagnosis | positive | positive | negative | positive | positive | negative |

In a further study, further diagnostic results demonstrate the prognostic utility of embodiments of the present invention by confirmation with actual diagnoses. Table 17 shows updated results from those patients and controls listed above:

TABLE 17
Summary of actual diagnoses corresponding (100%) to the blinded Cluster
Analysis of miRNA profiling

| | Serial sample No | | | | | | |
|---|---|---|---|---|---|---|---|
| | 1 | 2 | 3 | 5 | 6 | 7 | 8 |
| Blinded | D1 | D2 | D3 | D4 | D5 | D6 | D7 |
| Code | | | | | | | |
| Actual | Lung | Lung | Lung | Normal | Normal | Lung | Lung Cancer |
| Diagnosis | Cancer | Cancer | Cancer/ | Non- | Non- | Cancer | |
| | | | Treatment- | smoker | smoker | | |
| | | | Controlled | | | | |
| Micro RNA | Positive | Positive | Negative | Negative | Negative | Positive | Positive |
| Status | | | | | | | |
| Sex | M | M | M | M | M | M | M |

Although not shown in Table 17, serial sample nos. 4 and 9 were control subjects (blinded codes C1 and C2), both normal male smokers, with negative status from miRNA expression profile clustering.

In particular, the results for D3 as seen in FIG. 14 and Table 17 indicate that the treatment administered to D3 was effective. Upon treatment, the miRNA expression profile became less similar to the D1 and D2 cluster, as seen in FIG. 14. Therefore, in one embodiment, miRNA expression profile clustering may be used to monitor the effectiveness of treatment of subjects with lung cancer. Before treatment, the miRNA expression profile of a subject with lung cancer will be more similar, and will be grouped with, expression profiles from known lung cancers. After successful treatment, the miRNA expression profile will change to be less similar to the lung cancer profile, and more similar to normal profiles.

Exemplary embodiments of the present invention are described in the following Examples, which are set forth to aid in the understanding of the invention, and should not be construed to limit in any way the scope of the invention as defined in the claims which follow thereafter.

### Example 1

### RNA Extraction from Cell Lines

A TaqMan® MicroRNA Cell-to-C_{T}™ Kit (Applied Biosystems Inc., Foster City, Calif., USA) was used to extract RNA from the cell lines in accordance with the manufacturer's instructions. Briefly, 5×10⁵ cultured cells were plated into 96-well plates for six hours to allow attachment, and then washed with 4° C. phosphate buffered saline. 49 μl of Lysis Solution and 1 μl DNase I (provided by the supplier) was added into each well, and cells were incubated for eight minutes at room temperature. 5 μl of Stop Solution (provided by the supplier) was added to the lysate and incubated for two minutes at room temperature to inactivate the lysis reagents so that they would not inhibit reverse transcription (RT) or polymerase chain reaction (PCR). All cell lysates were stored on ice for less than two hours or at −70° C. for subsequent RT.

### Example 2

### Collection and Homogenization of Sputum

Sputum samples were collected, stored at 4° C., and processed within one week of collection. The sputum was separated from saliva. A 200 μl sample of sputum was transferred into a 1.5 ml nuclease-free tube. 400 μl of Sputolysin™ solution (0.1 mg/mL, Sigma, Canada) was added to the tube, vortexed until the viscous sputum was lysed, and incubated at 37° C. for 30 minutes. The homogenized sputum was stored at −20° C. until processed for RNA extraction.

### Example 3

### RNA Extraction from Sputum

1000 μL of Trizol™ (Invitrogen, USA) was added to the sputum sample tube (containing 200 μl of homogenized sputum), mixed with a pipet to re-suspend the sample, and vortexed for 20 seconds. 200 μl of chloroform was added, and the mixture was vortexed for 20 seconds and left at room temperature for five minutes. The tube was centrifuged for 15 minutes at 4° C. to separate the sample into an aqueous phase and a red organic phase. The aqueous phase (approximately 500 μl) was carefully transferred into a fresh 1.5 ml tube. 4 μl of glycogen co-precipitant (Ambion Inc., Austin, Tex., USA) and 5000 μl of isopropyl were added, gently mixed, and left at room temperature for twenty minutes. The tube was then centrifuged at 12000 rpm for 10 minutes, and for 15 minutes at 4° C. to co-precipitate RNA with glycogen. The RNA was carefully removed, washed with 75% ethanol at 4° C., and centrifuged at 8000 rpm at 4° C. for two minutes. RNA washing was repeated twice. The RNA was dissolved in 105 μl of nuclease-free water and stored at −20° C. until processed for reverse transcription and RT-PCR. To determine the RNA concentration and amount, 5 μl of RNA was diluted with 95 μl of nuclease-free water, and measured using a Beckman DU™ 7000 spectrophotometer (Beckman Coulter, Fullerton, Calif., USA). The amount of RNA (μg) in a 200 μl sample may range from about 0.5 μg to 3.0 μg. The ratio of adsorption at 260 nm over 280 nm should be greater than 1.0.

The concentration of RNA is calculated as:

RNA (μg/μl)=OD₂₆₀×20×40 [1]

The amount of RNA is calculated as:

Amount (μg)=OD₂₆₀×20×40×0.1 [2]

### Example 4

### Reverse Transcription

A TaqMan® MicroRNA Reverse Transcription Kit, primers, and StepOnePlus™ Real Time PCR system (Applied Biosystems Inc., Foster City, Calif., USA) were used for miRNA reverse transcription in accordance with the manufacturer's instructions. Briefly, the number of RT reactions was calculated and a RT Master Mix was assembled for all the reactions plus about 10% overage in a nuclease-free microcentrifuge tube on ice:

TABLE 17
RT Master Mix for single primer reactions

| Component | Each reaction |
|---|---|
| 10× RT Buffer | 1.5 μl |
| dNTP Mix | 0.15 μl |
| RNase Inhibitor | 0.19 μl |
| MultiScribe RT | 1.0 μl |
| Nuclease-free Water | 4.16 μl |
| Final Volume RT Master Mix | 7.0 μl |

The components were mixed gently and placed on ice. The RT Master Mix was distributed to nuclease-free PCR tubes. 3.0 μl of miRNA-specific primer was added to each aliquot of RT Master Mix followed by 5 μl of sample lysate for a final 15 μl reaction volume, followed by mixing and centrifugation at 1500 rpm for two minutes. The RT thermal cycler program was run according to the following settings:

TABLE 18
Thermal cycler settings for RT

| | Stage | Reps | Temperature | Time |
|---|---|---|---|---|
| Primer annealing | 1 | 1 | 16° C. | 30 min |
| Reverse transcription | 2 | 1 | 42° C. | 30 min |
| RT inactivation | 3 | 1 | 85° C. | 5 min |
| Hold | 4 | 1 | 4° C. | indefinite (50 min) |

The completed RT reactions were stored at −70° C. for subsequent quantitative RT-PCR.

### Example 5

### Quantitative RT-PCR

The comparative threshold cycle method (ΔΔC_{T}) was applied for miRNA profiling, using normal cell lines (GM38 or MRC-5) as reference samples and miR-92 as an endogenous control. A standard curve was generated to determine the limit of detection, reproducibility and variation. A TaqMan® Universal PCR Master Mix, Taqman® MicroRNA Assay, primers, probes, and StepOnePlus™ Real Time PCR system (Applied Biosystems Inc., Foster City, Calif., USA) were used for qRT-PCR in accordance with the manufacturer's instructions (Table 19).

TABLE 19
miRNA sequences, primers and probes

| | miRNA ref. No | Mature miRNA | TaqMan Assay | BioArray v9.2 |
|---|---|---|---|---|
| | | Seq v9.2 | Seq | Probe Seq |
| 1 | miR-17-3p | ACUGCAGUGA | ACUGCAGUGA | ACUGCAGUGA |
| | (ref. 1) | AGGCACUUGU | AGGCACUUGU | AGGCACUUGU |
| 2* | miR-17-5p | CAAAGUGCUUAC | CAAAGUGCUUAC | CAAAGUGCUUAC |
| | (ref. 5, 7) | AGUGCAGGUAGU | AGUGCAGGUAGU | AGUGCAGGUAGU |
| 3 | miR-19a | UGUGCAAAUCUA | UGUGCAAAUCUA | UGUGCAAAUCUA |
| | (ref. 4) | UGCAAAACUGA | UGCAAAACUGA | UGCAAAACUGA |
| 4* | miR-21 | UAGCUUAUCAG | UAGCUUAUCAG | UAGCUUAUCAG |
| | (ref. 1, 3, 4, 5, 6, 7) | ACUGAUGUUGA | ACUGAUGUUGA | ACUGAUGUUGA |
| 5 | miR-34a | UGGCAGUGUCUU | UGGCAGUGUCUU | UGGCAGUGUCUU |
| | (ref. 2) | AGCUGGUUGUU | AGCUGGUUGUU | AGCUGGUUGUU |
| 6* | miR-92 | UAUUGCACUUG | UAUUGCACUUG | UAUUGCACUUG |
| | (ref. 4) | UCCCGGCCUG | UCCCGGCCUG | UCCCGGCCUG |
| 7* | miR-143 | UGAGAUGAAGC | UGAGAUGAAGC | UGAGAUGAAGC |
| | (ref. 5, 6, 7) | ACUGUAGCUCA | ACUGUAGCUCA | ACUGUAGCUCA |
| 8* | miR-145 | GUCCAGUUUUCC | GUCCAGUUUUCC | GUCCAGUUUUCC |
| | (ref. 5, 6, 7) | CAGGAAUCCCUU | CAGGAAUCCCUU | CAGGAAUCCCUU |
| 9 | miR-14 [symbol] | UGAGAACUGAA | UGAGAACUGAA | UGAGAACUGAA |
| | (ref. 1) | UUCCAUGGGUU | UUCCAUGGGUU | UUCCAUGGGUU |
| 10 | mIR-152 | UCAGUGCAUGA | UCAGUGCAUGA | UCAGUGCAUGA |
| | (ref. 2) | CAGAACUUGGG | CAGAACUUGGG | CAGAACUUGGG |
| 11* | MiR-155 | UUAAUGCUAAU | UUAAUGCUAAU | UUAAUGCUAAU |
| | (ref. 1, 3, 4, 5, 7) | CGUGAUAGGGG | CGUGAUAGGGG | CGUGAUAGGGG |
| 12* | miR-182 | UUUGGCAAUGG | UUUGGCAAUGG | UUUGGCAAUGG |
| | (ref. 2) | UAGAACUCACA | UAGAACUCACA | UAGAACUCACA |
| 13 | miR-191 | CAACGGAAUCC | CAACGGAAUCC | CAACGGAAUCC |
| | (ref. 1, 4, 7) | CAAAAGCAGCU | CAAAAGCAGCU | CAAAAGCAGCU |
| 14 | miR-192 | CUGACCUAUGA | CUGACCUAUGA | CUGACCUAUGA |
| | (ref. 1) | AUUGACAGCC | AUUGACAGCC | AUUGACAGCC |
| 15 | miR-194 | UGUAACAGCAA | UGUAACAGCAA | UGUAACAGCAA |
| | (ref. 2) | CUCCAUGUGGA | CUCCAUGUGGA | CUCCAUGUGGA |
| 16 | miR-203 | GUGAAAUGUUU | GUGAAAUGUUU | GUGAAAUGUUU |
| | (ref. 1) | AGGACCACUAG | AGGACCACUAG | AGGACCACUAG |
| 17 | miR-205 | UCCUUCAUUCC | UCCUUCAUUCC | UCCUUCAUUCC |
| | (ref. 1, 2, 3, 4, 7) | ACCGGAGUCUG | ACCGGAGUCUG | ACCGGAGUCUG |
| 18* | miR-210 | CUGUGCGUGUG | CUGUGCGUGUG | CUGUGCGUGUG |
| | (ref. 1, 2, 4, 5, 7) | ACAGCGGCUGA | ACAGCGGCUGA | ACAGCGGCUGA |
| 19 | miR-212 | UAACAGUCUCC | UAACAGUCUCC | UAACAGUCUCC |
| | (ref. 1) | AGUCACGGCC | AGUCACGGCC | AGUCACGGCC |
| 20* | L [symbol] t-7a | UGAGGUAGUAG | UGAGGUAGUAG | UGAGGUAGUAG |
| | (ref. 4, 6) | GUUGUAUAGUU | GUUGUAUAGUU | GUUGUAUAGUU |

Endogenous control

| | |
|---|---|
| 1* | U6 |
| 2* | miR-92 |

Series number marked as * were miRNA selected in our past experiments
References:
(1) Rabinow its et al.;
(2) Rosenfeld et al.;
(3) Markou et al.;
(4) Barbarotto et al.;
(5) Stratagene (Agilent) PPT: High specificity miRNA qRT-PCR detection kit;
(6) Labourier, E. and Shingara, J. Applied Biosystem Technical Notes: microRNAs as potential diagnostic and prognostic markers of disease;
(7) Jay et al.
Note:
miR-17-92 cluster (miR-17-5p, miR-17-3p, miR-18a, miR-19a, miR-20a, miR-19b-1, miR-92-1) [symbol]
indicates data missing or illegible when filed

Briefly, the number of PCR assays was calculated and a PCR Cocktail was assembled for all the reactions plus about 10% overage in a nuclease-free microcentrifuge tube on ice:

TABLE 20
RT-PCR Cocktail for Single Primer RT Reactions

| Component | Each reaction |
|---|---|
| Taqman ® Master Mix (2X) | 10.0 μl |
| Taqman ® MicroRNA Assay | 1.0 μl |
| Nuclease-free water | 7.67 μl |
| Final volume RT-PCR Master Mix | 18.67 μl |

The components were mixed gently and placed on ice. The PCR Cocktail was distributed into PCR tubes. 1.33 μl of the RT product from Example 4 was added to each aliquot of PCR Cocktail for a final 20 μl reaction volume, followed by mixing and centrifugation at 1500 rpm for two minutes. The PCR instrument was run according to the following settings:

TABLE 21
PCR Cycling Conditions

| | Stage | Reps | Temperature | Time |
|---|---|---|---|---|
| Enzyme | 1 | 1 | 95° C. | 10 min |
| activation | | | | |
| PCR cycle | 2 | 40 | 95° C. | 15 sec |
| | | | 60° C. | 1 min |

### Example 6

### Cluster Analysis

SPSS 13.0 software (SPSS Inc., Chicago, Ill., USA) was used for hierarchical clustering according to the following steps:
a) Import data from an Excel™ spreadsheet to SPSS 13.0 software;
b) Select Analysis for Cliffify and open Hierarchical menu;
c) Select Case for cluster analysis and statistics, and plots for Display;
d) Open Statistic and select Agglomeration Schedule and Proximity Matrix;
e) Open Plot and select All Cluster;
f) Open Method and select “between-group linkage” as the cluster method, select “Pearson-correlation” as the convergence measure; and
g) Start cluster analysis and retrieve output results.

As will be apparent to those skilled in the art, various modifications, adaptations and variations of the foregoing specific disclosure can be made without departing from the scope of the invention claimed herein.

### REFERENCES

The following references are incorporated herein by reference (where permitted) as if reproduced in their entirety. All references are indicative of the level of skill of those skilled in the art to which this invention pertains.
Barbarotto, E.; Schmittgen, T. D. and Calin, G. A. (2008) MicroRNAs and cancer: profile, profile, profile. Int. J. Cancer. 122(5):969-77.
DesJardins, L. E. (2000) Isolation of M tuberculosis RNA from sputum. In Methods in Molecular Medicine, vol. 48: Antibiotic Resistance Methods and Protocols, ed. S. H. Gillespie, pp. 133-136.
Iorio, M. V.; Ferracin, M.; Liu, C. G.; Veronese, A.; Spizzo, R.; Sabbioni, S.; Magri, E.; Pedriali, M.; Fabbri, M.; Campiglio, M.; Ménard, S.; Palazzo, J. P.; Rosenberg, A.; Musiani, P.; Volinia, S.; Nenci, I.; Calin, G. A.; Querzoli, P.; Negrin, M. and Croce, C. M. (2005) MicroRNA gene expression deregulation in human breast cancer. Cancer Res. 65(16):7065-70.
Jay, C.; Nemunaitis, J.; Chen, P.; Fulgham, P. and Tong, A. W. (2007) miRNA profiling for diagnosis and prognosis of human cancer. DNA Cell Biol. 26(5):293-300. Review.
Johnson, S. M.; Grosshans, H.; Shingara, J.; Byrom, M.; Jarvis, R.; Cheng, A.; Labourier, E.; Reinert, K. L.; Brown, D. and Slack, F. J. (2005) RAS is regulated by the let-7 microRNA family. Cell 120(5):635-47.
Lacroix, J.; Becker, H. D.; Woerner, S. M.; Rittgen, W., Drings, P. and von Knebel Doebertiz, M. (2001) Sensitive detection of rare cancer cells in sputum and peripheral blood samples of patients with lung cancer by preprogrp-specific RT-PCR. Int. J Cancer 92:1-8.
Li, R.; Todd, N. W.; Qiu, Q.; Fan, T.; Zhao, R. Y.; Rodgers, WE., et al. (2007) Genetic deletions in sputum as diagnostic markers for early detection of stage I non-small cell lung cancer. Clin. Cancer Res. 13:482-487.
Markou, A.; Tsaroucha, E. G.; Kaklamanis, L.; Fotinou, M.; Georgoulias, V. and Lianidou, E. S. (2008) Prognostic value of mature microRNA-21 and microRNA-205 overexpression in non-small cell lung cancer by quantitative real-time RT-PCR. Clin. Chem. 54(10):1696-704. Epub 2008 Aug. 21.
Qiu, Q.; Todd, N. W.; Li, P.; Peng, H.; Liu, Z.; Yfantis, H. G.; Katz, R. L.; Stass, S. A. and Jiang, F. (2008) Magnetic enrichment of bronchial epithelial cells from sputum for lung cancer diagnosis. Cancer 114:275-283.
Rabinowits, G.; Gercel-Taylor, C.; Day, J. M.; Taylor, D. D. and Kloecker, G. H. (2009) Exosomal microRNA: a diagnostic marker for lung cancer. Clin. Lung Cancer 10(1):42-6.
Rosenfeld, N.; Aharonov, R.; Meiri, E.; Rosenwald, S.; Spector, Y.; Zepeniuk, M.; Benjamin, H.; Shabes, N.; Tabak, S.; Levy, A.; Lebanony, D.; Goren, Y.; Silberschein, E.; Targan, N.; Ben-An, A.; Gilad, S.; Sion-Vardy, N.; Tobar, A.; Feinmesser, M.; Kharenko, O.; Nativ, O.; Nass, D.; Perelman, M.; Yosepovich, A.; Shalmon, B.; Polak-Charcon, S.; Fridman, E.; Avniel, A.; Bentwich, I.; Bentwich, Z.; Cohen, D.; Chajut, A.; and Barshack, I. (2008) MicroRNAs accurately identify cancer tissue origin. Nat. Biotechnol. 26(4):462-9. Epub 2008 Mar. 23.
Thunnissen, F. B. (2003) Sputum examination for early detection of lung cancer. J. Clin. Pathol. 11:805-810.
Volinia, S.; Calin, G. A.; Liu, C. G.; Ambs, S.; Cimmino, A.; Petrocca, F.; Visone, R.; Iorio, M.; Roldo, C.; Ferracin, M.; Prueitt, R. L.; Yanaihara, N.; Lanza, G.; Scarpa, A.; Vecchione, A.; Negrini, M.; Harris, C. C. and Croce, C. M. (2006) A microRNA expression signature of human solid tumors defines cancer gene targets. Proc Natl Acad Sci USA 103(7):2257-61. Epub 2006 Feb. 3.
Yanaihara, N.; Caplen, N.; Bowman, E.; Seike, M.; Kumamoto, K.; Yi, M.; Stephens, R. M.; Okamoto, A.; Yokota, J.; Tanaka, T.; Calin, G. A.; Liu, C. G.; Croce, C. M.; and Harris, C. C. (2006) Unique microRNA molecular profiles in lung cancer diagnosis and prognosis. Cancer Cell 9(3):189-98.
Xie, Y.; Todd, N. W.; Liu, Z.; Zhan, M.; Fang, H.; Peng, H.; Alattar, M.; Deepak, J.; Stass, S. A. and Jiang, F. (2009) Altered miRNA expression in sputum for diagnosis of non-small cell lung cancer. Lung Cancer 2009 May 13. [Epub ahead of print]

What is claimed is:

1. A method of screening a subject for lung cancer comprising the steps of:
a) obtaining a sputum sample from the subject; and
b) determining a subject microRNA expression profile from the sputum sample;
c) determining whether or not the subject has lung cancer by determining a measure of similarity or dissimilarity of the subject expression profile to at least one known lung cancer microRNA expression profile and a known control microRNA expression profile;
wherein each of the subject and known expression profiles comprise the expression levels of at least two microRNAs.

2. The method of claim 1, wherein the lung cancer is a non-small cell lung carcinoma which is resistant to radiation and drugs, a non-small cell lung carcinoma which is sensitive to radiation and drugs, or a small cell lung carcinoma, or lung metastases originating from primary carcinomas of the breast, prostate, brain or other tissue.

3. The method of claim 1, wherein the measure of similarity or dissimilarity of the subject expression profile to at least one known lung cancer profile and a known control profile is determined by a statistical analysis.

4. The method of claim 3 wherein the statistical analysis comprises a hierarchical clustering step.

5. The method of claim 4 wherein the statistical analysis results in a cluster diagram or a dendogram.

6. The method of claim 1, wherein the at least two microRNAs comprise two or more of miR-21, miR-92, miR-143, miR-145, miR-155, miR-210, miR-17-5p, hsa-let-7a, hsa-miR-182, hsa-miR-205, or hsa-miR-372.

7. The method of claim 6, wherein the at least two microRNAS comprise two or more of miR-21, miR-155, miR-210, miR-143, or hsa-miR-372.

8. The method of claim 6, wherein the at least two microRNAS comprises miR-21, miR-155, miR-210, miR-143, and hsa-miR-372.

8. The method of claim 1 wherein the at least two microRNAs are grouped differently according to a cluster analysis.

9. The method of claim 1, wherein the at least two microRNAs comprise miR-145 and hsa-miR-205.

10. The method of claim 1, wherein any step of determining a microRNA expression profile comprises real-time quantitative RT-PCR detection.

11. A method of monitoring progress of a subject undergoing treatment for lung cancer, comprising the steps of determining a subject microRNA expression profile from a sputum sample from the subject obtained post-treatment and determining a measure of similarity or dissimilarity of the subject expression profile to at least one known lung cancer microRNA expression profile, a known control microRNA expression profile, or the subject microRNA expression profile pre-treatment.


SEQUENCE LISTING (62 sequences)

SEQ ID NO: 1 - RNA, 22 nt, Homo sapiens
uagcuuaucagacugauguuga

SEQ ID NO: 2 - RNA, 21 nt, Homo sapiens
uauugcacuugucccggccug

SEQ ID NO: 3 - RNA, 22 nt, Homo sapiens
ugagaugaagcacuguagcuca

SEQ ID NO: 4 - RNA, 24 nt, Homo sapiens
guccaguuuucccaggaaucccuu

SEQ ID NO: 5 - RNA, 22 nt, Homo sapiens
uuaaugcuaaucgugauagggg

SEQ ID NO: 6 - RNA, 22 nt, Homo sapiens
cugugcgugugacagcggcuga

SEQ ID NO: 7 - RNA, 24 nt, Homo sapiens
caaagugcuuacagugcagguagu

SEQ ID NO: 8 - RNA, 22 nt, Homo sapiens
ugagguaguagguuguauaguu

SEQ ID NO: 9 - RNA, 22 nt, Homo sapiens
uuuggcaaugguagaacucaca

SEQ ID NO: 10 - RNA, 22 nt, Homo sapiens
uccuucauuccaccggagucug

SEQ ID NO: 11 - RNA, 23 nt, Homo sapiens
aaagugcugcgacauuugagcgu

SEQ ID NO: 12 - DNA, 31 bp, Homo sapiens
gcaggggccatgctaatcttctctgtatwcg

SEQ ID NO: 13 - RNA, 20 nt, Homo sapiens
acugcagugaaggcacuugu

SEQ ID NO: 14 - RNA, 23 nt, Homo sapiens
ugugcaaaucuaugcaaaacuga

SEQ ID NO: 15 - RNA, 23 nt, Homo sapiens
uggcagugucuuagcugguuguu

SEQ ID NO: 16 - RNA, 22 nt, Homo sapiens
ugagaacugaauuccauggguu

SEQ ID NO: 17 - RNA, 22 nt, Homo sapiens
ucagugcaugacagaacuuggg

SEQ ID NO: 18 - RNA, 22 nt, Homo sapiens
caacggaaucccaaaagcagcu

SEQ ID NO: 19 - RNA, 21 nt, Homo sapiens
cugaccuaugaauugacagcc

SEQ ID NO: 20 - RNA, 22 nt, Homo sapiens
uguaacagcaacuccaugugga

SEQ ID NO: 21 - RNA, 22 nt, Homo sapiens
gugaaauguuuaggaccacuag

SEQ ID NO: 22 - RNA, 21 nt, Homo sapiens
uaacagucuccagucacggcc

SEQ ID NO: 23 - RNA, 20 nt, Artificial Sequence
  primer
acugcagugaaggcacuugu

SEQ ID NO: 24 - RNA, 24 nt, Artificial Sequence
  primer
caaagugcuuacagugcagguagu

SEQ ID NO: 25 - RNA, 23 nt, Artificial Sequence
  primer
ugugcaaaucuaugcaaaacuga

SEQ ID NO: 26 - RNA, 22 nt, Artificial Sequence
  primer
uagcuuaucagacugauguuga

SEQ ID NO: 27 - RNA, 23 nt, Artificial Sequence
  primer
uggcagugucuuagcugguuguu

SEQ ID NO: 28 - RNA, 21 nt, Artificial Sequence
  primer
uauugcacuugucccggccug

SEQ ID NO: 29 - RNA, 22 nt, Artificial Sequence
  primer
ugagaugaagcacuguagcuca

SEQ ID NO: 30 - RNA, 24 nt, Artificial Sequence
  primer
guccaguuuucccaggaaucccuu

SEQ ID NO: 31 - RNA, 22 nt, Artificial Sequence
  primer
ugagaacugaauuccauggguu

SEQ ID NO: 32 - RNA, 22 nt, Artificial Sequence
  primer
ucagugcaugacagaacuuggg

SEQ ID NO: 33 - RNA, 22 nt, Artificial Sequence
  primer
uuaaugcuaaucgugauagggg

SEQ ID NO: 34 - RNA, 22 nt, Artificial Sequence
  primer
uuuggcaaugguagaacucaca

SEQ ID NO: 35 - RNA, 22 nt, Artificial Sequence
  primer
caacggaaucccaaaagcagcu

SEQ ID NO: 36 - RNA, 21 nt, Artificial Sequence
  primer
cugaccuaugaauugacagcc

SEQ ID NO: 37 - RNA, 22 nt, Artificial Sequence
  primer
uguaacagcaacuccaugugga

SEQ ID NO: 38 - RNA, 22 nt, Artificial Sequence
  primer
gugaaauguuuaggaccacuag

SEQ ID NO: 39 - RNA, 22 nt, Artificial Sequence
  primer
uccuucauuccaccggagucug

SEQ ID NO: 40 - RNA, 22 nt, Artificial Sequence
  primer
cugugcgugugacagcggcuga

SEQ ID NO: 41 - RNA, 21 nt, Artificial Sequence
  primer
uaacagucuccagucacggcc

SEQ ID NO: 42 - RNA, 22 nt, Artificial Sequence
  primer
ugagguaguagguuguauaguu

SEQ ID NO: 43 - RNA, 20 nt, Artificial Sequence
  probe
acugcagugaaggcacuugu

SEQ ID NO: 44 - RNA, 24 nt, Artificial Sequence
  probe
caaagugcuuacagugcagguagu

SEQ ID NO: 45 - RNA, 23 nt, Artificial Sequence
  probe
ugugcaaaucuaugcaaaacuga

SEQ ID NO: 46 - RNA, 22 nt, Artificial Sequence
  probe
uagcuuaucagacugauguuga

SEQ ID NO: 47 - RNA, 23 nt, Artificial Sequence
  probe
uggcagugucuuagcugguuguu

SEQ ID NO: 48 - RNA, 21 nt, Artificial Sequence
  probe
uauugcacuugucccggccug

SEQ ID NO: 49 - RNA, 22 nt, Artificial Sequence
  probe
ugagaugaagcacuguagcuca

SEQ ID NO: 50 - RNA, 24 nt, Artificial Sequence
  probe
guccaguuuucccaggaaucccuu

SEQ ID NO: 51 - RNA, 22 nt, Artificial Sequence
  probe
ugagaacugaauuccauggguu

SEQ ID NO: 52 - RNA, 22 nt, Artificial Sequence
  probe
ucagugcaugacagaacuuggg

SEQ ID NO: 53 - RNA, 22 nt, Artificial Sequence
  probe
uuaaugcuaaucgugauagggg

SEQ ID NO: 54 - RNA, 22 nt, Artificial Sequence
  probe
uuuggcaaugguagaacucaca

SEQ ID NO: 55 - RNA, 22 nt, Artificial Sequence
  probe
caacggaaucccaaaagcagcu

SEQ ID NO: 56 - RNA, 21 nt, Artificial Sequence
  probe
cugaccuaugaauugacagcc

SEQ ID NO: 57 - RNA, 21 nt, Artificial Sequence
  probe
uguaacagcaacuccagugga

SEQ ID NO: 58 - RNA, 22 nt, Artificial Sequence
  probe
gugaaauguuuaggaccacuag

SEQ ID NO: 59 - RNA, 22 nt, Artificial Sequence
  probe
uccuucauuccaccggagucug

SEQ ID NO: 60 - RNA, 22 nt, Artificial Sequence
  probe
cugugcgugugacagcggcuga

SEQ ID NO: 61 - RNA, 21 nt, Artificial Sequence
  probe
uaacagucuccagucacggcc

SEQ ID NO: 62 - RNA, 22 nt, Artificial Sequence
  probe
ugagguaguagguuguauaguu